(19) United States
(12) Patent Application Publication
(10) Pub. No.: US 2026/0345749 A1
(43) Pub. Date: Nov. 5, 2026

(54) METHODS OF MAKING AND USING NANOPARTICLES FOR TREATMENT OF MICROBIAL BIOFILMS

(71) Applicant: Brown University, Providence, RI (US)

(72) Inventors: Anita Shukla, East Greenwich, RI (US); Yingying Wang, Providence, RI (US); Zhaowei Jiang, Providence, RI (US)

(21) Appl. No.: 19/478,919

(22) PCT Filed: Apr. 26, 2024
(86) PCT No.: PCT/US2024/026546

§ 371 Date: Oct. 27, 2025

Related U.S. Application Data
(60) Provisional application No. 63/462,582, filed on Apr. 28, 2023.

(51) Int. Cl. A61K 38/46; A61K 9/51; A61P 31/04

(52) U.S. Cl. CPC A61K 38/465; A61K 9/5146; A61K 9/5169; A61P 31/04; C12Y 301/21001

(57) ABSTRACT

A method of making and using nanoparticles that target biofilm are provided.

### STATEMENT OF GOVERNMENT INTERESTS

This invention was made with government support under N00014-22-1-2336 awarded by the Office of Naval Research. The government has certain rights in the invention.

### FIELD

The present invention relates in general to the design and use of nanoparticles to penetrate microbial biofilms, such as bacterial and/or fungal biofilms and release a payload, such as an antimicrobial, an antibacterial, and/or an antifungal agent. The nanoparticles also include an antibiofilm agent, which can degrade the biofilm.

### BACKGROUND

Biofilms are structured microbial communities comprising microbial cells (bacteria and/or fungi) embedded in a self-produced extracellular polymeric substance (EPS) matrix composed of polysaccharides, proteins, extracellular DNA (eDNA) and other components. See R. Joseph, A. Naugolny, M. Feldman, I. M. Herzog, M. Fridman and Y. Cohen, Journal of the American Chemical Society, 2016, 138, 754-757. Biofilm-associated microbial infections are considered as one of the greatest threats to health worldwide. It is estimated that around 17 million new biofilm-associated infections arise and lead to up to 550,000 deaths annually in the United States alone. See R. J. Worthington, J. J. Richards and C. Melander, Org. Biomol. Chem., 2012, 10, 7457-7474. Biofilms can be formed from a single species of microbes, such as bacterial biofilms or fungal biofilms. Polymicrobial biofilms are formed from more than one species of microbes that can include both bacteria and fungi. Polymicrobial biofilm infections have a higher risk of death (about 70%) than infections caused by a single species of microbe (23%). See Zago C E, Silva S, Sanitá PV, Barbugli P A, Dias M I, Lordello V B, et al. Dynamics of biofilm formation and the interaction between Candida albicans and methicillin-susceptible (MSSA) and resistant Staphylococcus aureus (MRSA). PLoS One, 2015, 10, 1-15. Biofilms are involved in various conditions including dental caries, urinary tract infections, burn wound infections, diabetic foot ulcers. See Y. Liu, L. Shi, L. Su, H. C. van der Mei, P. C. Jutte, Y. Ren and H. J. Busscher, Chem. Soc. Rev., 2019, 48, 428-446.

S. aureus biofilm infections are characterized by their high prevalence and remarkable resilience in clinical settings. See Lister, J. L., Horswill, A. R. Staphylococcus aureus biofilms: Recent developments in biofilm dispersal. Frontiers in Cellular and Infection Microbiology, 2014, 4, 1-9; Munteanu, N. et al. Carta a Ludwig Kugelmann (12 de abil 1871). Microbial Pathogenesis, 2017, 8, 329-340. Biofilms formed by S. aureus have been implicated in a wide range of infections, including but not limited to medical device-related infections, wound infections, and chronic infections. See Idrees, M.; Sawant, S., Karodia, N., Rahman, A. Staphylococcus aureus biofilm: Morphology, genetics, pathogenesis and treatment strategies. International Journal of Environmental Research and Public Health, 2021, 18, 7602; Otto, M. Staphylococcal biofilms. Gram-Positive Pathogens, 2019, 699-711. For example, S. aureus has been reported to be responsible for 18.9% of surgical site infections (SSIs), which often results in a high mortality rate. See S lowik, R., Ko lpa, M., Wa laszek, M., R'oza'nska, A., Jagiencarz-Starzec, B., Zie'nczuk, W., Kawik, Wolak, Z., Wojkowska-Mach, J., Epidemiology of surgical site infections considering the NHSN standardized infection ratio in hip and knee arthro-plasties. International Journal of Environmental Research and Public Health, 2020, 17, 1-10; Swolana, D., Wojtyczka, R. D., Activity of Silver Nanoparticles against Staphylococcus spp. International Journal of Molecular Sciences, 2022, 23. These biofilms are complex microbial communities encased within an EPS, providing enhanced protection and resistance against host immune responses and antimicrobial agents. See Periasamy, S., Joo, H. S., Duong, A. C., Bach, T. H. L., Tan, V. Y., Chatterjee, S. S., Cheung, G. Y., Otto, M., How Staphylococcus aureus biofilms develop their characteristic structure. Proceedings of the National Academy of Sciences of the United States of America, 2012, 109, 1281-1286. The ability of S. aureus biofilms to persist and thrive is attributed to various factors, such as the formation of a protective barrier, altered gene expression, quorum sensing mechanisms, and the presence of persister cells. Eradicating S. aureus biofilms through traditional antibiotic therapy presents a formidable challenge due to several interrelated factors. The biofilm EPS exhibits altered physiology and heightened resistance to conventional antimicrobial treatments and host immune responses. See Sharma, K., Singh, A. P., Antibiofilm Effect of DNase against Single and Mixed Species Biofilm. Foods, 2018, 7(3): 42; Costerton, J. W., Stewart, P. S., Greenberg, E. P., Bacterial biofilms: A common cause of persistent infections. Science, 1999, 284, 1318-1322. The EPS affords the biofilm with heightened resistance to antimicrobial. See Cepas, V., L'opez, Y., Muñoz, E., Rolo, D., Ardanuy, C., Mart'1, S., Xercavins, M., Horcajada, J. P., Bosch, J., Soto, S. M., Relationship between Biofilm Formation and Antimicrobial Resistance in Gram-Negative Bacteria. Microbial Drug Resistance, 2019, 25, 72-79; Bowler, P., Murphy, C., Wolcott, R., Biofilm exacerbates antibiotic resistance: Is this a current oversight in antimicrobial stewardship? Antibiotic resistance, 2020, 9(1): 162; Hall, C. W., Mah, T. F., Molecular mechanisms of biofilm-based antibiotic resistance and tolerance in pathogenic bacteria. FEMS Microbiology Reviews, 2017, 41, 276-301. Additionally, the biofilms protect the embedded microorganism from altered pH, osmolarity and mechanical forces, blocking the penetration of antibiotics and immune cells. The persistence and resilience of S. aureus biofilm infections present a significant challenge in clinical practice, necessitating innovative strategies for their effective management.

In addition to their complex structure and heightened resistance mechanisms, S. aureus biofilm infections contribute significantly to the global challenge of antimicrobial resistance (AMR). AMR, which occurs when microorganisms develop mechanisms to nullify the effects of antimicrobial drugs, is a natural phenomenon that poses one of the greatest threats to global public health. Many antimicrobials and antibiofilm agents have been developed with the advancement of biology and medicine to combat infections caused by biofilms and mitigate the spread of AMR. See Mishra, R., Kumari Panda, A., De Mandal, S., Shakeel, M., Singh Bisht, S., Khan, J., Natural Anti-biofilm Agents: Strategies to Control Biofilm-Forming Pathogens. Front Microbiol, 2020, 11: 566325.; Li, X.-H., Lee, J.-H. Antibiofilm agents: A new perspective for antimicrobial strategy. Journal of Microbiology, 2017, 55, 753-766; Ventola, C. L. The Antibiotic Resistance Crisis: Part 1: Causes and Threats. Pharmacy and Therapeutics, 2015, 40, 277; Hernando-Amado, S., Coque, T. M., Baquero, F., Mart'inez, J. L. Antibiotic Resistance: Moving From Individual Health Norms to Social Norms in One Health and Global Health. Frontiers in Microbiology, 2020, 11: 1914; Sabtu, N., Enoch, D. A., Brown, N. M. Antibiotic resistance: what, why, where, when and how? British Medical Bulletin, 2015, 116, 105-113. However, the persistent nature of biofilms and their ability to confer resistance complicate treatment outcomes and necessitate the continual development of innovative therapeutic strategies. See Prestinaci, F., Pezzotti, P., Pantosti, A. Antimicrobial resistance: a global multifaceted phenomenon. Pathog Glob Health, 2015, 109(7):309-18. Recognizing the urgency of addressing AMR, the Global Research on Antimicrobial Resistance (GRAM) report estimated that in 2019 alone, 4.95 million infection-related deaths were associated with AMR. See Murray, C. J. et al. Global burden of bacterial antimicrobial resistance in 2019: a systematic analysis. The Lancet, 2022, 399, 629-655.

The most commonly used method for treatment of biofilm-related infections includes oral or intravenous administration of United States Food and Drug Administration (FDA) approved antibiotics. See C. Deusenbery, Y. Wang and A. Shukla, ACS Infectious Diseases, 2021, 7, 695-720. However, the recalcitrance of biofilms toward antibiotics impairs the therapeutic effect of this traditional therapy. See D. Davies, Nature Reviews Drug Discovery, 2003, 2, 114-122, and D. Lebeaux, J.-M. Ghigo and C. Beloin, Microbiol Mol Biol Rev., 2014, 78, 510-543. In fact, high antibiotic doses (up to 10-1000 times greater than concentrations effective against planktonic bacteria) and prolonged treatment times are usually required for the treatment of biofilm-associated bacterial infections, which can exacerbate toxicity and resistance. See T.-F. Mah, B. Pitts, B. Pellock, G. C. Walker, P. S. Stewart and G. A. O'Toole, Nature, 2003, 426, 306-310 and P. Bowler, C. Murphy, R. Wolcott, Antimicrobial Resistance and Infection Control, 2020, 9, 162.

With the advancement of biology and medicine, many antimicrobials and antibiofilm alternatives have recently been developed. See Lojewska, E., Sakowicz, T. An Alternative to Antibiotics: Selected Methods to Combat Zoonotic Foodborne Bacterial Infections. Current Microbiology, 2021, 78, 4037-4049; Mishra, R., Panda, A. K., De Mandal, S., Shakeel, M., Bisht, S. S., Khan, J. Natural Anti-biofilm Agents: Strategies to Control Biofilm-Forming Pathogens. Front Microbiol, 2020, 11:566325; Roy, R., Tiwari, M., Donelli, G., Tiwari, V. Strategies for combating bacterial biofilms: A focus on anti-biofilm agents and their mechanisms of action. Virulence, 2018, 9, 522-554; Dostert, M., Trimble, M. J., Hancock, R. E. Antibiofilm peptides: overcoming biofilmrelated treatment failure. RSC Advances, 2021, 11, 2718-2728; Deusenbery, C., Wang, Y., Shukla, A. Recent Innovations in Bacterial Infection Detection and Treatment. ACS Infectious Diseases, 2021, 7, 695-720; Karygianni, L., Ren, Z., Koo, H., Thurnheer, T. Biofilm Matrixome: Extracellular Components in Structured Microbial Communities. Trends in Microbiology, 2020, 28, 668-681. Antibiotic alternatives commonly include repurposed drugs, antimicrobial peptides and polymers, nucleic acids, small molecules, bacteriophages, nanoparticles (NPs), etc. See Deusenbery, C., Wang, Y., Shukla, A. Recent Innovations in Bacterial Infection Detection and Treatment. ACS Infectious Diseases, 2021, 7, 695-720. Several antibiofilm agents (e.g., deoxyribonuclease I (DNase), polysaccharide hydrolases, and proteases) are effective to degrade EPS and disperse biofilm biomass. See Karygianni, L., Ren, Z., Koo, H., Thurnheer, T. Biofilm Matrixome: Extracellular Components in Structured Microbial Communities. Trends in Microbiology, 2020, 28, 668-681. However, the direct use of drugs is low efficacy because of the lack of targeting drug delivery and the difficulty of penetrating the biofilms. See Anderl, J. N., Franklin, M. J., Stewart, P. S. Role of antibiotic penetration limitation in Klebsiella pneumoniae biofilm resistance to ampicillin and ciprofloxacin. Antimicrobial Agents and Chemotherapy, 2000, 44, 1818-1824; Stewart, P. S. Mechanisms of antibiotic resistance in bacterial biofilms. International Journal of Medical Microbiology, 2002, 292, 107-113. To address this problem, NPs were used to load both antimicrobials and antibiofilm agents to improve the effect of infection treatment. See Liu, Y., Shi, L., Su, L., Van Der Mei, H. C., Jutte, P. C., Ren, Y., Busscher, H. J. Nanotechnology-based antimicrobials and delivery systems for biofilm-infection control. Chem. Soc. Rev., 2019, 48, 428; Koo, H., Allan, R. N., Howlin, R. P., Stoodley, P., Hall-Stoodley, L. Targeting microbial biofilms: current and prospective therapeutic strategies. Nature Reviews Microbiology, 2017, 15, 740-755. For example, Tasia et al. developed a mesoporous silica NP to load with both DNase and silver nanoparticles (AgNPs) and investigated the biofilm eradication effect. See Tasia, W., Lei, C., Cao, Y., Ye, Q., He, Y., Xu, C. Enhanced eradication of bacterial biofilms with DNase I-loaded silver-doped mesoporous silica nanoparticles. Nanoscale, 2020, 12. Their results showed enhanced antibiofilm effects for Escherichia coli and Streptococcus mutans biofilms. In another example, Weldrick et al. designed protease-functionalized antibiotic nanocarriers to combat wound-related pathogenic bacterial biofilms. See Weldrick, P. J., Hardman, M. J., Paunov, V. N. Enhanced Clearing of Wound-Related Pathogenic Bacterial Biofilms Using Protease-Functionalized Antibiotic Nanocarriers. ACS Appl. Mater. Interfaces, 2019, 11, 47, 43902-43919. A 6-fold decrease in the biofilm mass was found, indicating the excellent biofilm eradication ability of protease. Although these studies successfully delivered antimicrobials and antibiofilm agents to biofilm, developing drug delivery systems with targeted delivery and controlled release is still challenging.

In particular, smart responsive NPs can selectively react with specific stimuli (e.g. pH and enzyme), leading to targeted delivery of antimicrobial agents to achieve improved therapeutic efficacy. See Ismail, E. A., Devnarain, N., Govender, T., Omolo, C. A. Stimuli-responsive and biomimetic delivery systems for sepsis and related complications. Journal of Controlled Release, 2022, 352, 1048-1070; Yang, Y., Jiang, X., Lai, H., Zhang, X. Smart Bacteria-Responsive Drug Delivery Systems in Medical Implants. Journal of Functional Biomaterials, 2022, 13(4): 173. The use of gelatin for the formulation of nanocarriers has been investigated by many research groups. See Ofokansi, K., Winter, G., Fricker, G., Coester, C. Matrix-loaded biodegradable gelatin nanoparticles as new approach to improve drug loading and delivery. Eur J Pharm Biopharm, 2010, 76(1): 1-9.; Leo, E., Vandelli, M. A., Cameroni, R., Forni, F. Doxorubicin-loaded gelatin nanoparticles stabilized by glutaraldehyde: Involvement of the drug in the cross-linking process. International Journal of Pharmaceutics, 1997, 155, 75-82; Teng, K. Y., Lu, Z., Wientjes, M. G., Au, J. L. Formulating paclitaxel in nanoparticles alters its disposition. Pharmaceutical Research, 2005, 22, 867-874; Truong-le, V. L., Walsh, S. M., Schweibert, E., Mao, H.-q., Guggino, W. B., August, J. T., Leong, K. W. Gene Transfer by DNA—Gelatin Nanospheres. Arch Biochem Biophys, 1999, 361(1): 47-56; Kaul, G., Amiji, M. Cellular interactions and in vitro DNA transfection studies with poly(ethylene glycol)-modified gelatin nanoparticles. Journal of Pharmaceutical Sciences, 2005, 94, 184-198; Kaul, G.; Amiji, M. Tumor-targeted gene delivery using poly(ethylene glycol)-modified gelatin nanoparticles: In Vitro and in Vivo studies. Pharmaceutical Research, 2005, 22, 951-961; Balthasar, S., Michaelis, K., Dinauer, N., Von Briesen, H., Kreuter, J., Langer, K. Preparation and characterisation of antibody modified gelatin nanoparticles as drug carrier system for uptake in lymphocytes. Biomaterials, 2005, 26, 2723-2732; Dinauer, N., Balthasar, S., Weber, C., Kreuter, J., Langer, K., Von Briesen, H. Selective targeting of antibody-conjugated nanoparticles to leukemic cells and primary T-lymphocytes. Biomaterials, 2005, 26, 5898-5906. Gelatin polymers are naturally occurring biopolymers formed from collagen degradation and can be further hydrolyzed by gelatinases. Gelatinases are proteases produced by bacteria, capable of hydrolyzing gelatin into amino acids. It has been previously demonstrated that biopolymer coated gelatin-based nanoparticles have excellent biofilm penetration and retention properties in Vibrio vulnificus biofilms. See Wang, Y., Shukla, A. Bacteria-responsive biopolymer-coated nanoparticles for biofilm penetration and eradication. Biomaterials Science, 2022, 2831-2843.

With recent advances in nanotechnology, nanomaterials have shown promise as new antimicrobials and delivery systems to combat biofilm-associated microbial infection. See S. Fulaz, S. Vitale, L. Quinn and E. Casey, Trends in Microbiology, 2019, 27, 915-926. Numerous nanomaterials have been reported as promising antibiotic alternatives such as metal-based nanoparticles (NPs) (e.g. silver nanoparticles (see Y. Dong, H. Zhu, Y. Shen, W. Zhang and L. Zhang, PLoS One, 2019, 14, e0222322 and X. Dai, Q. Guo, Y. Zhao, P. Zhang, T. Zhang, X. Zhang and C. Li, ACS Applied Materials & Interfaces, 2016, 8, 25798-25807), gold nanoparticles (see X. Yang, J. Yang, L. Wang, B. Ran, Y. Jia, L. Zhang, G. Yang, H. Shao and X. Jiang, ACS Nano, 2017, 11, 5737-5745) and palladium nanoparticles (see G. Fang, W. Li, X. Shen, J. M. Perez-Aguilar, Y. Chong, X. Gao, Z. Chai, C. Chen, C. Ge and R. Zhou, Nature Communications, 2018, 9, 129), cationic polymeric NPs (see A. Gupta, R. F. Landis, C.-H. Li, M. Schnurr, R. Das, Y.-W. Lee, M. Yazdani, Y. Liu, A. Kozlova and V. M. Rotello, Journal of the American Chemical Society, 2018, 140, 12137-12143; X. K. Liu, Lihong, H. Wang, P. K. J. Tan, W. Fan, S. S. Venkatraman, L. Li and Y.-Y. Yang, Nature Nanotechnology, 2009, 4, 457-463; and A. Ivanova, K. Ivanova, J. Hoyo, T. Heinze, S. Sanchez-Gomez and T. Tzanov, ACS Applied Materials & Interfaces, 2018, 10, 3314-3323), and quantum dots (see C. M. Courtney, S. M. Goodman, J. A. McDaniel, N. E. Madinger, A. Chatterjee and P. Nagpal, Nature Materials, 2016, 15, 529-534). However, methods and materials are needed for the treatment of bacterial and/or fungal infections which can overcome the resistance provided by the bacterial and/or fungal biofilms.

### SUMMARY

Aspects of the present disclosure are directed to nanoparticles and methods of using such nanoparticles to treat microbial biofilms, such as bacterial biofilm and the bacteria within, which contributes to bacterial resistance, and fungal biofilm and the fungus within, which contributes to fungal resistance. According to one aspect, the nanoparticles penetrate the microbial biofilm, such as bacterial biofilm or fungal biopfilm. After the nanoparticle has penetrated the microbial biofilm, the nanoparticle releases a payload agent. The payload agent may be an antimicrobial, which is toxic to the microbe within the microbial biofilm, such as bacteria or fungus. According to one aspect, the microbes die, and therefore cease to produce the microbial biofilm. As a result, the microbial biofilm is destroyed or otherwise eradicated. According to an additional aspect, the nanoparticles include an antibiofilm agent, such as a nuclease, which degrades extracellular DNA (eDNA), such as that forming the microbial biofilm. In this manner, the biofilm itself is degraded, destroyed or otherwise eradicated.

The present disclosure describes methods of treating an individual in need thereof for a microbial infection, such as a bacterial infection or fungal infection. The nanoparticles described herein are administered to an individual and penetrate a microbial biofilm within the individual. The nanoparticles release an antimicrobial within the microbial biofilm thereby treating the individual for the microbial infection. The nanoparticles include a nuclease which degrades the extracellular DNA (eDNA) of the microbial biofilm, such as a bacterial biofilm or fungal biofilm.

According to one aspect, the nanoparticle includes a core. The core includes gelatin. The core includes the payload agent, such as an antibiotic, a bactericide, or other antimicrobial agent. Due to the production of gelatinases by many common biofilm pathogens and at infection sites, gelatin nanoparticles degrade at the site of infection to release the payload agent, such as an antimicrobial or other therapeutic. According to one embodiment, the core includes or is coated with a nuclease which degrades extracellular DNA (eDNA) of a bacterial biofilm. According to one embodiment, a pH-degradable coating, such as a poly(ethylene glycol) (PEG) derivative, is conjugated to the gelatin nanoparticles to protect nuclease-coated gelatin nanoparticles. According to one embodiment, the core is responsive to a gelatinase produced by the biofilm pathogen, such as at an infection site. Accordingly, the core can be said to be a gelatinase-responsive core.

The nanoparticle may include a layer or coating on or otherwise surrounding the core. The layer or coating may be responsive to a first condition or environment of the biofilm which exposes the core to a gelatinase which then degrades the core and releases the payload agent.

Additional aspects of the present disclosure are directed to nanoparticles and methods of using such nanoparticles to treat microbial biofilm and the microbes within, which contributes to microbial resistance. According to one aspect, the nanoparticles penetrate the microbial biofilm. After the nanoparticle has penetrated the microbial biofilm, the nanoparticle releases a payload agent. The payload agent may be an antimicrobial, which is toxic to the microbes within the microbial biofilm. According to one aspect, the microbes die, and therefore cease to produce the microbial biofilm. As a result, the microbial biofilm is destroyed or otherwise eradicated. According to an additional aspect, the nanoparticles include an antibiofilm agent, such as a nuclease, a polysaccharide hydrolase, and/or a protease. The nuclease degrades the extracellular DNA (eDNA), the polysaccharide hydrolase degrades the polysaccharide, and the protease degrades the proteins, all of which are components of the biofilm matrix. In this manner, the biofilm itself is degraded, destroyed or otherwise eradicated.

The present disclosure describes methods of treating an individual in need thereof for a microbial infection. The nanoparticles described herein are administered to an individual and penetrate a microbial biofilm within the individual. The nanoparticles release an antimicrobial within the microbial biofilm thereby treating the individual for the microbial infection. The microbial infection includes a bacterial infection, a fungal infection, or both a bacterial infection and a fungal infection. The nanoparticles include an antibiofilm agent, such as a nuclease, a polysaccharide hydrolase, and/or a protease. The antibiofilm agent degrades the components of the biofilm matrix, such as the extracellular DNA (eDNA), the polysaccharide, and the proteins.

According to one aspect, the nanoparticle includes a core. The core includes gelatin. The core includes the payload agent, such as an antibiotic, a bactericide, an antifungal agent, or other antimicrobial agent. Due to the production of gelatinases by many common biofilm pathogens and at infection sites, gelatin nanoparticles degrade at the site of infection to release the payload agent, such as an antimicrobial or other therapeutic. According to one embodiment, the core includes or is coated with a nuclease which degrades extracellular DNA of a bacterial and/or fungal biofilm. According to one embodiment, a pH-degradable coating, such as a poly(ethylene glycol) (PEG) derivative, is conjugated to the gelatin nanoparticles to protect nuclease-coated gelatin nanoparticles. According to one embodiment, the core is responsive to a gelatinase produced by the biofilm pathogen, such as at an infection site. Accordingly, the core can be said to be a gelatinase-responsive core.

The nanoparticle may include one or more layers or coatings on or otherwise surrounding or conjugated to the core. The layer(s) or coating(s) may be responsive to a first condition or environment of the biofilm. When the layer(s) or coating(s) are degraded, the antibiofilm agent along with the core are exposed, and the gelatinase within the biofilm in turn degrades the core and releases the payload antimicrobial agent.

It is to be understood that the term nanoparticle includes a particle having a core and may have one or more layers. According to one aspect, the nanoparticle includes a core and one or more, two or more or a plurality of layers.

According to one aspect, the nanoparticles described herein are effective to inhibit or otherwise prevent microbial biofilm formation. According to one aspect, the nanoparticles described herein are effective to inhibit or prevent growth of existing microbial biofilms. According to one aspect, the nanoparticles described herein are effective to reduce the presence of existing biofilms or otherwise destroy or eradicate existing biofilms. The microbial can include bacteria and/or fungi, and other microbes known to one skilled in the art.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 depicts synthesis and characterization of NPs. Panel a. demonstrates proposed mechanism of R-GNPs. Panel b. depicts a schematic synthesis approach for making a gelatin nanoparticle (GNP) having silver nanoparticles therein (Ag-GNP) and having a nuclease (DNase I) attached to the surface of the nanoparticle core and also having a pH-degradable material attached to the surface of the nanoparticle core. The complete nanoparticle is referred to as “P-D-Ag-GNP” or “R-GNP” (a complete responsive nanoparticle). The terms “P-D-Ag-GNP” and “R-GNP” are used herein interchangeably. Panels c-e. depict TEM images of GNPs (c), Ag-GNPs (d) and R-GNPs (e), respectively. Images are representative of at least three independent experiments per NP formulation. Scale bars are 100 nm. Panel f. demonstrates the hydrodynamic diameter (black bars) and polydispersity index (grey bars) of the responsive NPs in 1×PBS at pH 7.4. Results are shown as mean±standard deviation. Statistical significance (a, b, c, d, e, f, h, i, k, 1 indicate p<0.0001, g indicates p<0.01, and j indicates p<0.001) between groups was determined using two-way ANOVA with Tukey's post hoc analysis; n=3.

FIGS. 2A-2E depict the visualization and analysis of eDNA in Biofilm Formations and Supernatant pH Measurements, eDNA visualization in S. aureus (SA) 25923 biofilm (FIG. 2A); S. aureus (SA) Xen29 biofilms (FIG. 2B); S. epidermidis (SE) 12228 biofilms (FIG. 2C); and P. aeruginosa (PA) PA01 biofilm (FIG. 2D), demonstrating eDNA presence indicative of biofilm matrix development. (FIG. 2E) Comparative graph of pH values for the supernatants from each bacterial species' biofilm at 24 hours, reflecting potential acidity of the biofilm environment for bacteria of interest. Statistical significance between conditions is indicated by matching using two-way ANOVA with multiple comparisons with Dunnett's test (* indicates p<0.1, ** indicates p<0.01, *** indicates p<0.001 and **** indicates p<0.0001), with n=3.

FIG. 3 depicts a synthetic pathway of the pH cleavable co-polymer (mPEG-b-i-bPEI).

FIG. 4 depicts FTIR spectra of p-formylbenzoic acid, mPEG, mPEG-benzoic acid, bPEI and mPEG-benzoic-imine-bPEI, corresponding to panels a-e from top to bottom.

FIG. 5 depicts 1H NMR of PEG-benzoic-imine-PEI copolymer mPEG-benzoic acid (panel a) and mPEG-benzoic-imine-bPEI (panel b) in D₂O.

FIG. 6 depicts zeta-potential measurements of NP formulations. Results are shown as mean±standard deviation. All studies have at least n=3.

FIG. 7 depicts EDX spectrum analysis showing major peak of silver nanoparticles at 3 eV, corresponding to silver nanoparticles.

FIG. 8 depicts the stimuli-responsiveness of NP formulations. FTIR spectrum of GNP (panel a) and R-GNP (panel b) at pH 7.4. FTIR spectrum of R-GNP at pH 4.0 (panel c). Agarose gel electrophoresis image of NP formulation treated DNA plasmid at pH 4.0 (panel d). ICP-OES analysis of Ag+ release from Ag-GNP (panel e) and R-GNP (panel f) under different pHs. The results of panels f and g are shown as mean±standard deviation. All studies have at least n=3.

FIG. 9 depicts the FTIR spectra of GNP (panel a) and R-GNP (panel b).

FIG. 10 depicts the MIC (panel a) and MBC (panel b) antimicrobial efficacy of commercially available silver nanoparticle (AgNP) treated against S. aureus 25923, S. epidermidis 12228 and P. aeruginosa PA01. Statistical significance between conditions is indicated by matching using two-way ANOVA with multiple comparisons with Dunnett's test (* indicates p<0.1, ** indicates p<0.01, *** indicates p<0.001 and **** indicates p<0.0001), with n=3.

FIG. 11 depicts minimal inhibitory (MIC) and bactericidal concentrations (MBC) of DNase I and PEG-b-i-PEI against S. aureus 25923, S. epidermidis 12228 and P. aeruginosa PA01. Panel a shows MIC of DNase I against S. aureus 25923, S. epidermidis 12228 and P. aeruginosa PA01. Panel b shows MIC of PEG-b-i-PEI against S. aureus 25923, S. epidermidis 12228 and P. aeruginosa PA01. Panel c shows MBC of DNase I against S. aureus 25923, S. epidermidis 12228 and P. aeruginosa PA01. And Panel d shows MBC of PEG-b-i-PEI against S. aureus 25923, S. epidermidis 12228 and P. aeruginosa PA01. Statistical significance between conditions is indicated by matching using two-way ANOVA with multiple comparisons with Dunnett's test (* indicates p<0.1, ** indicates p<0.01, *** indicates p<0.001 and **** indicates p<0.0001), with n=3.

FIG. 12 depicts MIC and MBC of NP formulations against selected bacterial strains: (panel a) MIC of gelatin nanoparticles (GNP), (panel b) MIC of silver-loaded GNP (Ag-GNP), (panel c) MIC of blank responsive GNP (P-D-GNP), (panel d) MBC of GNP, (panel e) MBC of Ag-GNP, and (panel f) MBC of P-D-GNP. The bacterial strains tested include S. aureus 25923, S. epidermidis 12228, and P. aeruginosa PA01. Statistical significance between conditions is indicated by matching using two-way ANOVA with multiple comparisons with Dunnett's test (* indicates p<0.1, ** indicates p<0.01, *** indicates p<0.001 and **** indicates p<0.0001), with n=3.

FIG. 13 depicts dose-response curves illustrating the antibacterial activity and biofilm inhibition by R-GNPs across different bacterial strains: (Panel a) MIC curves depicting the percentage of normalized bacterial growth at escalating R-GNP concentrations. (Panel b) MBC curves displaying the colony-forming units per milliliter (CFU/mL) following R-GNP treatment. (Panel c) MBIC curves showing the relative changes in biofilm biomass after exposure to R-GNPs. Statistical significance between conditions is indicated by matching using two-way ANOVA with multiple comparisons with Dunnett's test (* indicates p<0.1, ** indicates p<0.01, indicates p<0.001 and *** indicates p<0.0001), with n=3.

FIG. 14 depicts the MBIC (panel a) and MBEC (panel b) antibiofilm efficacy of commercially available silver nanoparticle (AgNP) treated against S. aureus 25923, S. epidermidis 12228 and P. aeruginosa PA01. Statistical significance between conditions is indicated by matching using two-way ANOVA with multiple comparisons with Dunnett's test (* indicates p<0.1, ** indicates p<0.01, *** indicates p<0.001 and **** indicates p<0.0001), with n=3.

FIG. 15 depicts the antibiofilm efficacy of DNase MBIC and MBEC (panels a and c) and co-polymer MBIC and MBEC (panels b and d) treated against S. aureus 25923, S. epidermidis 12228 and P. aeruginosa PA01. Statistical significance between conditions is indicated by matching using two-way ANOVA with multiple comparisons with Dunnett's test (* indicates p<0.1, ** indicates p<0.01, *** indicates p<0.001 and **** indicates p<0.0001), with n=3.

FIG. 16 depicts biofilm eradication efficacy upon exposure to various NP formulations. Biofilm eradication ability upon GNP (panel a), Ag-GNP (panel b) and R-GNP (panel c) treatments with different concentrations. Panel d depicts SEM images of biofilm samples treated with various NP formulations at 32 μg/mL and an equal volume of 1×PBS served as a positive control. Panel e depicts R-GNP biofilm eradication efficacy against S. aureus clinical isolates. Results are shown as mean±standard deviation. Statistical significance is indicated by matching using two-way ANOVA with multiple comparisons with Dunnett's test (* indicates p<0.1, ** indicates p<0.01, *** indicates p<0.001 and **** indicates p<0.0001), with n=3. Images are representative of at least three independent experiments per NP formulation. Scale bars are 200 nm.

FIG. 17 depicts minimal biofilm inhibitory concentrations of NP formulations against S. aureus 25923, S. epidermidis 12228 and P. aeruginosa PA01. (Panel a) MBIC of gelatin nanoparticles (GNP), (Panel b) MBIC of silver-loaded GNP (Ag-GNP), (Panel c) MBIC of blank responsive GNP (P-D-GNP), (Panel d) MBEC of gelatin nanoparticles (GNP), (Panel e) MBEC of silver-loaded GNP (Ag-GNP), (Panel f) MBEC of blank responsive GNP (P-D-GNP). Statistical significance between conditions is indicated by matching using two-way ANOVA with multiple comparisons with Dunnett's test (* indicates p<0.1, ** indicates p<0.01, *** indicates p<0.001 and **** indicates p<0.0001), with n=3.

FIG. 18 depicts S. aureus biofilm images upon treatment with NP formulations. Panels a-c: LIVE/DEAD stained S. aureus biofilm post-treatment with GNP (panel a), Ag-GNP (panel b) and (panel c) R-GNP. Scale bars are 50 μm (white) and 20 μm (yellow). The red color in the images indicates dead cells with membrane damage, and the green color indicates live cells with intact membranes. Panels d-e: Ruby biofilm matrix-stained S. aureus biofilm treated with GNP (panel d), Ag-GNP (panel e), P-DGNP (panel f) and R-GNP (panel g). The red color in the confocal images indicates biofilm matrix stained with FilmTracer™ SYPRO™ Ruby Biofilm Matrix Stain. All images are representative of at least three independent experiments per NP formulation.

FIG. 19 depicts images of biofilm eradication efficacy upon S. aureus 25923, S. epidermidis 12228 or P. aeruginosa PA01 cells exposed to various NP formulations and concentrations. SEM of biofilm samples treated with 32 μg/mL GNP, Ag-GNP and R-GNP treatments. Images are representative of at least three independent experiments per NP formulation. Scale bars are 200 nm.

FIG. 20 depicts graphs of in vitro cytotoxicity and hemolysis of NP formulations. Panel a. Cytotoxicity of NP formulations with NIH 3T3 and panel b. HUVEC cells evaluated by cell count kit 8 (CCK-8) assay; And panel b. Human red blood cell hemolysis upon incubation with NP formulations. Results are shown as mean±standard deviation with n=3. Statistical significance between conditions is indicated by matching using two-way ANOVA with multiple comparisons with Dunnett's test (* indicates p<0.1, ** indicates p<0.01, *** indicates p<0.001 and indicates p<0.0001), with n=3.

FIG. 21 depicts a schematic and images for utilizing ex vivo porcine skin infection to investigate R-GNP biofilm eradication efficacy. Panel a shows a schematic of the process of utilizing ex vivo porcine skin infection to investigate R-GNP biofilm eradication efficacy. Panel b shows a representative SEM image of infected procine skin treated with GNP. Panel c shows a representative SEM image of infected porcine skin treated with Ag-GNP. All experiments were performed with at least three samples; scale bar=50 μm.

FIG. 22 depicts images of ex vivo antibiofilm efficacy of R-GNP. Porcine skin explants were wounded and infected with bioluminescent S. aureus 25923. Panels a-c show representative SEM images of skin explants post-infection, post-infection with treatment and treatment only (left to right); Representative images are shown with white scale bar=50 μm and yellow scale bar=20 μm. Panel d depicts MIC results with various R-GNP concentration treating C. albican SC5314 planktonic cells. Panel e depicts MBIC results with various R-GNP concentration treating C. albican SC5314 biofilm samples. All experiments were performed with at least three samples. Statistical significance between conditions is indicated by matching using two-way ANOVA with multiple comparisons with Dunnett's test (* indicates p<0.1, ** indicates p<0.01, *** indicates p<0.001 and **** indicates p<0.0001), with n=3.

FIG. 23 depicts a schematic for utilizing in vivo murine skin infection to investigate R-GNP biofilm eradication efficacy.

FIG. 24 depicts results for in vivo efficacy of R-GNP-mediated antibiofilm activity. 7-week-old mice were wounded and infected with bioluminescent S. aureus Xen29. (Panel a) Bacterial burden was monitored using IVIS imaging with the total in vivo bioluminescent signal. Representative IVIS bioluminescent signals superimposed on a grayscale photographic image of the mice, illustrating in vivo biofilm disruption following R-GNP treatments; Scale bar=5 mm. (Panel b) The bioluminescent signal was quantified and normalized with control background and represented as normalized flux changes %. Results are shown as mean±standard deviation. (Panel c) Monitoring of CFU during the course of infection (Panel d). All experiments were performed with at least three samples (i.e., 3 male, 3 female).

### DETAILED DESCRIPTION

The present disclosure provides methods of designing and using nanoparticles to deliver a payload, such as an antibiotic, antimicrobial, antibacterial, and/or antifungal agent, and also an antibiofilm agent such as a nuclease for the degradation of extracellular DNA characteristic of a microbial biofilm. According to certain aspects, the microbial biofilms include bacterial or fungal biofilms. The microbial biofilms can also include polymicrobial biofilms, which contain both bacteria and fungi. According to aspects described herein, nanoparticles are designed and used to treat microbial biofilms in a manner to prevent and/or inhibit and/or eradicate microbial biofilms, including polymicrobial biofilms. The nanoparticles are designed to be responsive to one or more enzymes or one or more conditions of the biofilms so as to cause the nanoparticle to degrade and release an antimicrobial agent. According to an additional aspect, the nanoparticles include an antibiofilm agent. The antibiofilm agent serves to degrade the various components of the biofilm matrix. For example, the antibiofilm includes but are not limited to a nuclease, a polysaccharide hydrolase, and/or a protease. The nuclease such as DNase I degrades the extracellular DNA (eDNA), the polysaccharide hydrolase degrades the polysaccharide, and the protease degrades the proteins, all of which are components of the biofilm matrix. In this manner, the biofilm itself is degraded, destroyed or otherwise eradicated.

According to certain aspects, the present disclosure provides gelatin nanoparticles as an antimicrobial and an antibiofilm agent delivery vehicle. Due to the production of gelatinases by many common biofilm pathogens and at infection sites, the gelatin nanoparticles respond to the gelatinases in the biofilm and degrade at the site of infection to release loaded therapeutics. According to the present disclosure, gelatin nanoparticle formulations are provided for the stimuli-responsive release of antimicrobial agents. The gelatin nanoparticles include silver nanoparticles (AgNPs) within the gelatinase-responsive core. The silver nanoparticles act as a broad-spectrum antibiotic and kill bacteria and/or fungi in the biofilm. In addition, an antibiofilm agent, e.g. a nuclease, such as, deoxyribonuclease I (DNase I), is attached or conjugated to the gelatin nanoparticles. DNase I exhibits antibiofilm properties by degrading extracellular DNA, a major component of the biofilm EPS. To protect DNase stability until it encounters the biofilm microenvironment, a pH-degradable agent, such as a poly(ethylene glycol) (PEG) derivative, is conjugated to the DNase-coated GNPs to form a coating. The pH-degradable coating is degraded or shed in the presence of low pH at the biofilm site, thereby exposing the DNase I. The present disclosure provides a dual-stimuli responsive (i.e., pH and enzyme-responsive) nanoparticle to treat microbial biofilms by locally delivering a combination treatment of antibiofilm (nuclease) and antimicrobial agents (silver nanoparticles).

The present disclosure recognizes that the microenvironment of microbial infection sites is unlike that in normal tissue in terms of the concentration or composition of various substances. See D. Hu, Y. Deng, F. Jia, Q. Jin and J. Ji, ACS Nano, 2020, 14, 347-35; C. Wang, W. Zhao, B. Cao, Z. Wang, Q. Zhou, S. Lu, L. Lu, M. Zhan and X. Hu, Chemistry of Materials, 2020, 32, 7725-7738; Y. Liu, A. Lin, J. Liu, X. Chen, X. Zhu, Y. Gong, G. Yuan, L. Chen and J. Liu, ACS Applied Materials & Interfaces, 2019, 11, 26590-26606; D. Alkekhia, H. Safford, S. Shukla, R. Hopson and A. Shukla, Chem. Commun., 2020, 56, 11098-11101; D. Pornpattananangkul, L. Zhang, S. Olson, S. Aryal, M. Obonyo, K. Vecchio, C.-M. Huang and L. Zhang, Journal of the American Chemical Society, 2011, 133, 4132-4139.

For example, lower pH is often found at bacterial and/or fungal infection sites. See S. Fulaz, D. Hiebner, C. H. N. Barros, H. Devlin, S. Vitale, L. Quinn and E. Casey, ACS Applied Materials & Interfaces, 2019, 11, 32679-32688. The localized acidic biofilm microenvironment results from the production of acidic by-products during bacterial metabolism. See A. M. Scharnow, A. E. Solinski and W. M. Wuest, Med. Chem. Commun., 2019, 10, 1057-1067. Overexpression of proteases also occurs at the infection sites. See G.-B. Qi, D. Zhang, F.-H. Liu, Z.-Y. Qiao and H. Wang, Advanced Materials, 2017, 29, 1703461; L.-L. Li, H.-L. Ma, G.-B. Qi, D. Zhang, F. Yu, Z. Hu and H. Wang, Advanced Materials, 2016, 28, 254-262; X. Wang, J. Wu, P. Li, L. Wang, J. Zhou, G. Zhang, X. Li, B. Hu and X. Xing, ACS Applied Materials & Interfaces, 2018, 10, 34905-34915. For example, bacterial and/or fungal gelatinases contribute to biofilm formation and virulence through degradation of a broad range of host substrates. See L. R. Thurlow, V. C. Thomas, S. Narayanan, S. Olson, S. D. Fleming and L. E. Hancock, Infection and Immunity, 2010, 78, 4936-4943. Another class of common enzyme found at the infection sites are hyaluronidases which are virulence factors that are involved in the invasion, and penetration of tissues in bacterial infection. See W. L. Hynes and S. L. Walton, FEMS Microbiology Letters, 2000, 183, 201-207.

According to the present disclosure, nanoparticles are designed based on these biofilm environment features using materials responsive to these biofilm environment features, such as a gelatin core and a pH-degradable conjugation or a coating. According to one aspect, a nanoparticle is provided which includes a core. The core may include a treatment agent, such as an antibiotic and or an antimicrobial agent. The core may have an antibiotic, such as silver nanoparticles, and a material responsive to an enzyme which degrades the material, such as gelatin responsive to a gelatinase. When the core is degraded, the treatment material is released. According to one aspect, the nanoparticle includes an antibiofilm agent. The antibiofilm agent can include one or more enzymes that degrade the various components of the biofilm matrix. For example, the antibiofilm agent can include a nuclease, such as DNase I, attached or conjugated to the surface of the core (which may be in the form of a layer or coating) which degrades extracellular DNA (eDNA) of the bacterial and/or fungal biofilm. The nanoparticle may include a pH-degradable material attached to the surface of the core (which may be in the form of a layer or coating). At the site of the bacterial and/or fungal biofilm, the pH-degradable material is responsive to low pH and is degraded exposing the nuclease which then degrades extracellular DNA (eDNA) present in the biofilm.

According to one aspect, the present disclosure is directed to the targeted destruction of bacterial biofilms wherein a nanoparticle including a core and an antibiotic, and a nuclease are delivered to the bacterial biofilm. The nuclease is exposed so as to degrade extracellular DNA of the bacterial biofilm and an antibiotic is released to treat bacteria present at the bacterial biofilm. Degradation of the gelatin core will increase loaded antibiotic release from the gelatin core, leading to efficient bacteria death and biofilm eradication.

Gelatin nanoparticles used herein may be those described in drug delivery systems such as P. S. O. Victoria and Speiser, U.K Patent GB1516348, 1978 and R. C. Oppenheim, J. J. Marty and P. Speiser, U.S. Pat. No. 4,107,288, each of which is hereby incorporated herein by reference in its entirety. Various surface modifications which may be useful to incorporate targeting agents, increase nanoparticle NP stability, and control loading and/or release are described in A. Lin, Y. Liu, X. Zhu, X. Chen, J. Liu, Y. Zhou, X. Qin and J. Liu, ACS Nano, 2019, 13, 13965-13984; L.-L. Li, J.-H. Xu, G.-B. Qi, X. Zhao, F. Yu and H. Wang, ACS Nano, 2014, 8, 4975-4983; J. Su, R. Zhang, Y. Lian, Z. Kamal, Z. Cheng, Y. Qiu and M. Qiu, Pharmaceutics, 2019, 11, 93; S. Kirar, N. S. Thakur, J. K. Laha and U. C. Banerjee, ACS Applied Bio Materials, 2019, 2, 4202-4212; S. Balthasar, K. Michaelis, N. Dinauer, H. v. Briesen, J. Kreuter and K. Langer, Biomaterials, 2005, 26, 2723-2732; T. G. Shutava, S. S. Balkundi, P. Vangala, J. J. Steffan, R. L. Bigelow, J. A. Cardelli, D. P. O'Neal and Y. M. Lvov, ACS Nano, 2009, 3, 1877-1885; X.-h. Tian, F. Wei, T.-x. Wang, D. Wang, J. Wang, X.-n. Lin, P. Wang and L. Ren, Materials Letters, 2012, 68, 94-96 each of which is hereby incorporated herein by reference in its entirety.

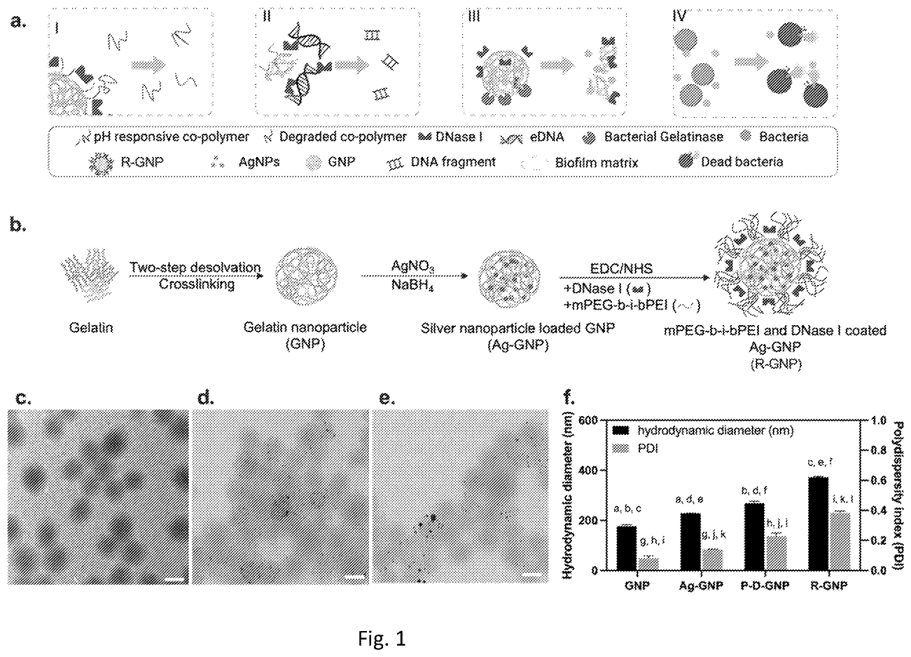

The present disclosure provides that, in the presence of gelatinases, GNPs will be hydrolyzed, resulting in the release of loaded antimicrobial agents (FIG. 1, panel a). GNPs will also be coated with antibiofilm agents to facilitate efficient penetration and eradication of bacterial and/or fungal biofilms. Additional layers of pH-triggered polymer on the outermost layer of the NPs enable pH stimuli-triggered exposure of the coated antibiofilm agents. AgNPs possess a broad spectrum of antibacterial properties. See Le Quay, B., Stellacci, F. Antibacterial activity of silver nanoparticles: A surface science insight. Nano Today, 2015, 10, 339-354; Feng, Q. L., Wu, J., Chen, G. Q., Cui, F. Z., Kim, T. N., Kim, J. O. A mechanistic study of the antibacterial effect of silver ions on Escherichia coli and Staphylococcus aureus. Journal of Biomedical Materials Research, 2000, 52, 662-668; Siddiqi, K. S., Husen, A., Rao, R. A. A review on biosynthesis of silver nanoparticles and their biocidal properties. Journal of Nanobiotechnology, 2018, 16 (1): 14; Marambio-Jones, C., Hoek, E. M. A review of the antibacterial effects of silver nanomaterials and potential implications for human health and the environment. Journal of Nanoparticle Research, 2010, 12, 1531-1551. The antibacterial and antifungal mechanism of AgNPs is due to their ability to damage cell membranes, produce reactive oxygen species and interrupt deoxyribonucleic acid replication by releasing Ag ions. See Yin, I. X., Zhang, J., Zhao, I. S., Mei, M. L., Li, Q., Chu, C. H. The antibacterial mechanism of silver nanoparticles and its application in dentistry. International Journal of Nanomedicine, 2020, 15, 2555-2562. By utilizing AgNPs as an alternative to traditional antibiotics, this approach contributes to the reduction of antibiotic resistance. DNase was chosen as an exemplary antibiofilm agent due to its ability to degrade extracellular DNA (eDNA) within the biofilm EPS. See Fleming, D., Rumbaugh, K. P. Approaches to dispersing medical biofilms. Microorganisms, 2017, 5. As biofilm eradication relies on debridement of the EPS and killing the encapsulated microbes, enzymes depolymerize the biofilm matrix, making them excellent candidates to disrupt biofilm EPS. See Chen, Z., Haiwei, J., Liu, C., Ing, W., Wang, Z., Qu, X. A Multinuclear Metal ComplexBased DNase-Mimetic Artificial Enzyme: Matrix Cleavage for Combating Bacterial Biofilms, Angew Chem Int Ed Engl, 2016, 55 (36): 10732-6; Fuxman Bass, J. I., Russo, D. M., Gabelloni, M. L., Geffner, J. R., Giordano, M., Catalano, M., Ngeles Zorreguieta, A., Trevani, A. S. Extracellular DNA: A Major Proinflammatory Component of Pseudomonas aeruginosa Biofilms, J Immunol, 2010, 184 (11): 6386-95. The present disclosure contemplates using enzymes that depolymerize various components, e.g., the extracellular DNA, polysaccharides, and proteins, of the biofilm matrix, as antibiofilm agents. In an exemplary embodiment, to protect DNase stability until it encounters the biofilm microenvironment, a pH-degradable poly(ethylene glycol) (PEG) derivative was conjugated to the DNase-coated GNPs. In this copolymer, the imine linkage is the pH-responsive linker which can be hydrolyzed into aldehyde (ketone) and amine in pH<6.5 environments. See Deirram, N., Zhang, C., Kermaniyan, S. S., Johnston, A. P., Such, G. K. pH-Responsive Polymer Nanoparticles for Drug Delivery. Macromolecular Rapid Communications, 2019, 40, 1-23; Park, W., Park, S. J., Shin, H., Na, K. Acidic Tumor pH-Responsive Nanophotomedicine for Targeted Photodynamic Cancer Therapy. Journal of Nanomaterials 2016, 5, 1-8. The acidic biofilm environment ensures the pH-triggered polymer release and subsequential exposure of DNase to biofilm matrixes. See Hu, D., Deng, Y., Jia, F., Jin, Q., Ji, J. Surface Charge Switchable Supramolecular Nanocarriers for Nitric Oxide Synergistic Photodynamic Eradication of Biofilms. ACS Nano, 2020, 14, 347-359. DNase will be exposed and destabilize the EPS to facilitate the penetration of antimicrobial agents. Bacterial and or fungal gelatinases will degrade the GNP core and lead to the release of the loaded AgNPs, which will lead to efficient bacteria and/or fungi inhibition and biofilm eradication. According to exemplary embodiments, the mechanism and the dispersing effects towards the biofilms of these NPs were tested on the established S. aureus, P. aeruginosa, S. epidermidis and C. albicans biofilms.

In one embodiment, the present disclosure provides engineered gelatin nanoparticles (GNPs) that exhibit pH- and bacterial and/or fungal enzyme-triggered drug release, resulting in antibiofilm efficacy. It is hypothesized that the GNPs are degraded by bacterial and/or fungal gelatinases at the site of infection, releasing the therapeutic payload. Silver nanoparticles (AgNPs) were synthesized within the gelatinase-responsive core, serving as a broad-spectrum antibiotic alternative. To enhance antibiofilm properties, deoxyribonuclease I (DNase) was conjugated to the GNPs to form the complete responsive GNP formulation (R-GNP). This complete responsive GNP is also referred to herein as “P-D-Ag-GNP”. As disclosed herein, R-GNP and P-D-Ag-GNP are used interchangeably and refers to the complete, dual-responsive GNP (responding to both pH and gelatinase). DNase degrades extracellular DNA, a major component of the biofilm matrix. To ensure stability of the DNase until it encounters the biofilm, a pH-degradable poly(ethylene glycol) derivative was conjugated to the DNase-coated GNPs. It is hypothesized that the low pH at the biofilm site would cause the coating to be shed, exposing the DNase.

In exemplary embodiments, dynamic light scattering (DLS) measurements showed an increase in NP hydrodynamic diameter with the addition of AgNP in the GNPs and a further increase with surface functionalization. Transmission electron microscopy (TEM) confirmed the successful formation of AgNPs within the GNPs, while inductively coupled plasma-optical emission spectrometry (ICP-OES) suggested 17.7±0.1% (w/w) Ag ions within the R-GNPs. The conjugation of DNase was confirmed by monitoring plasmid degradation upon incubation with the R-GNPs via gel electrophoresis, while the conjugation and pH-responsiveness of the co-polymer were verified via Fourier-transform infrared spectroscopy, following overnight incubation of NPs in an acidic pH. Enzyme-linked immunosorbent assay (ELISA) suggested that 5.48±0.31% (w/w) DNase I was conjugated onto the NP core. Antibiofilm efficacy of the NP formulations was evaluated using Staphylococcus aureus 25923, Staphylococcus epidermidis 12228, and Pseudomonas aeruginosa PA01. S. aureus and S. epidermidis which form acidic biofilms, while P. aeruginosa forms basic biofilms, and only S. aureus and P. aeruginosa produce gelatinases. The R-GNPs led to significant eradication of S. aureus and S. epidermidis biofilms, with an 80% reduction in biofilm biomass at 32 μg/mL for S. aureus and S. epidermidis, and 128 μg/mL for P. aeruginosa. In a murine superficial S. aureus biofilm infection model, treatment with R-GNPs led to a significant reduction in bacterial burden compared to non-treated controls. The present disclosure thus describes a dual stimuli-responsive nanoparticle as a treatment of bacterial and/or fungal biofilms. The ability to disrupt biofilms and enhance the penetration of therapeutic agents provides for treatment against biofilm-related infections, addressing a critical challenge in the management of chronic bacterial and/or fungal diseases.

According to the present disclosure, an exemplary bacterium to be treated is S. aureus. According to the present disclosure, an exemplary fungus to be treated is Candida albicans. It is to be understood that the present disclosure is not limited to S. aureus and/or C. albicans. Instead, the nanoparticles can be designed to treat and/or deliver an antibiotic to biofilms created by any bacteria and/or fungi where the microenvironment of the biofilm is an acidic pH and optionally where the bacteria and/or fungi produce a gelatinase. It is contemplated that administration includes systemic and topical administration methods.

### I. Exemplary Bacteria and Fungi

Bacteria according to the present disclosure includes any bacteria which creates a biofilm and into which nanoparticles can be introduced and which generates an acidic environment (such as a low pH). Exemplary bacteria may also produce a gelatinase. It is to be understood that the basic concepts of the present disclosure described herein are not limited by bacteria type. Bacteria according to the present disclosure may include one or members of the species Vibrio, Clostridium, Escherichia, Rhodococcus, Pseudomonas, Bacillus, Lactobacillus Saccharomyces, Acinetobacter, Staphylococcus, Enterobacter, Klebsiella and Enterococcus.

Exemplary genus and species of bacteria cells for use in the methods described herein include Acetobacter aurantius, Acinetobacter bitumen, Actinomyces israelii, Agrobacterium radiobacter, Agrobacterium tumefaciens, Anaplasma Anaplasma phagocytophilum, Azorhizobium caulinodans, Azotobacter vinelandii, viridans streptococci, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides, Bacteroides fragilis, Bacteroides gingivalis, Bacteroides melaninogenicus (also referred to as Prevotella melaninogenica), Bartonella, Bartonella henselae, Bartonella quintana, Bordetella, Bordetella bronchiseptica, Bordetella pertussis, Borrelia burgdorferi, Brucella abortus, Brucella melitensis, Brucella suis, Burkholderia, Burkholderia mallei, Burkholderia pseudomallei, Burkholderia cepacia, Calymmatobacterium granulomatis, Campylobacter, Campylobacter coli, Campylobacter fetus, Campylobacter jejuni, Campylobacter pylori, Chlamydia, Chlamydia trachomatis, Chlamydophila Chlamydophila pneumoniae (also known as Chlamydia pneumoniae) Chlamydophila psittaci (also known as Chlamydia psittaci), Clostridium, Clostridium botulinum, Clostridium difficile, Clostridium perfringens (also known as Clostridium welchii), Clostridium tetani, Corynebacterium, Corynebacterium diphtheriae, Corynebacterium fusiforme, Coxiella burnetii, Ehrlichia chaffeensis, Enterobacter cloacae, Enterococcus, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma, Mycoplasma fermentans, Mycoplasma genitalium, Mycoplasma hominis, Mycoplasma penetrans, Mycoplasma pneumoniae, Neisseria, Neisseria gonorrhoeae, Neisseria meningitidis, Pasteurella, Pasteurella multocida, Pasteurella tularensis, Peptostreptococcus, Porphyromonas gingivalis, Prevotella melaninogenica (also known as Bacteroides melaninogenicus), Pseudomonas aeruginosa, Rhizobium radiobacter, Rickettsia, Rickettsia prowazekii, Rickettsia psittaci, Rickettsia quintana, Rickettsia rickettsii, Rickettsia trachomae, Rochalimaea, Rochalimaea henselae, Rochalimaea quintana, Rothia dentocariosa, Salmonella, Salmonella enteritidis, Salmonella typhi, Salmonella typhimurium, Serratia marcescens, Shigella dysenteriae, Staphylococcus, Staphylococcus aureus, Staphylococcus epidermidis, Stenotrophomonas maltophilia, Streptococcus Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema, Treponema pallidum, Treponema denticola, Vibrio, Vibrio cholerae, Vibrio comma, Vibrio parahaemolyticus, Vibrio vulnificus, Wolbachia, Yersinia, Yersinia enterocolitica, Yersinia pestis, and Yersinia pseudotuberculosis, and other genus and species known to those of skill in the art.

Fungi according to the present disclosure includes any fungi which creates a biofilm and into which nanoparticles can be introduced and which generates an acidic environment (such as a low pH). Exemplary fungi may also produce a gelatinase. It is to be understood that the basic concepts of the present disclosure described herein are not limited by fungi type. Fungi according to the present disclosure may include one or members of the Candida species, such as Candida albicans, and Cryptococcus neoformans, Cryptococcus gattii, Rhodotorula species, Aspergillus fumigatus, Malassezia pachydermatis, Histoplasma capsulatum, Paracoccidioides brasiliensis, Pneumocystis species, Coccidioides immitis, Fusarium species, Saccharomyces cerevisiae, Trichosporon asahii, Mucorales and Blastoschizomyces and the like, and other genus and species known to those of skill in the art.

### II. Microbial Conditions or Environments

As described herein, the exemplary bacteria S. aureus or fungal C. albicans creates a biofilm. According to certain aspects, the biofilm can be formed by a combination of bacteria and fungi. The biofilm is characterized by an acidic pH and optionally the presence of proteases, such as gelatinases. An exemplary biofilm may be characterized by both an acidic pH and the presence of a gelatinase. As described herein, nanoparticles are described that respond to the bacterial and/or fungal enzymes and pH. For example, a material which can be degraded in an acidic pH can be used. A material that can be degraded by a gelatinase can be used. One of skill can design the nanoparticle core and materials attached thereto to be responsive to one or more conditions of the bacterial and/or fungal biofilm.

### III. Nanoparticles Having Cores and Layers

Exemplary nanoparticles as described herein have a diameter of between 1 and less than 1000 nanometers, between 1 and 500 nm, between 1 and 250 nm, between 1 and 200 nm, between 1 and 150 nm, between 1 and 100 nm although nanoparticles can also have diameters suitable for the particular application. Nanoparticles can have diameters above 100 nanometers and below 1 nanometer. Nanoparticles are usually present as a distribution of diameters.

### IV. Exemplary Antibiotic, Antimicrobial Agents and Antifungal Agents

Active agents according to the present disclosure include antibiotics, antimicrobials, antibacterial agents, antifungal agents, and other agents known to be toxic to bacteria and/or fungi. An antibiotic is generally understood to refer to an antimicrobial substance active against bacteria. Antibiotics, antimicrobials, antibacterial agents and/or antifungal agents are used to fight bacterial and/or fungal infections, and antibiotic and antimicrobial medications are widely used in the treatment and prevention of such infections. They may either kill or inhibit the growth of bacteria and/or fungi, or prevent bacteria and/or fungi from replicating. Antibiotics are commonly classified based on their mechanism of action, chemical structure, or spectrum of activity. Most of these antibiotics target bacterial functions or growth processes. Those that target the bacterial cell wall (penicillins and cephalosporins) or the cell membrane (polymyxins), or interfere with essential bacterial enzymes (rifamycins, lipiarmycins, quinolones, and sulfonamides) have bactericidal activities. Protein synthesis inhibitors (macrolides, lincosamides, and tetracyclines) are usually bacteriostatic (with the exception of bactericidal aminoglycosides). Further categorization is based on their target specificity. “Narrow-spectrum” antibiotics target specific types of bacteria, such as gram-negative or gram-positive, whereas broad-spectrum antibiotics affect a wide range of bacteria.

An exemplary alternative antimicrobial includes silver, such as provided by silver nitrate (AgNO₃). Silver is a well-documented antimicrobial and has a long history of use in wound care, which has been shown to kill bacteria, fungi and certain viruses. It is the positively charged silver ions (Ag+) that possess the antimicrobial effect. Silver ions target microorganisms through several different modes of action.

Exemplary antimicrobial agents include amoxicillin, doxycycline, cephalexin, ciprofloxacin, clindamycin, metronidazole, azithromycin, sulfamethoxazole, trimethoprim, clavulanate, and levofloxacin.

Exemplary antimicrobial agents include Vancomycin, Teicoplanin, Linezolid, Daptomycin, Trimethoprim/sulfamethoxazole, Doxycycline, Ceftobiprole, Ceftaroline, Clindamycin, Dalbavancin, Fusidic acid, Mupirocin, Omadacycline, Oritavancin, Tedizolid, Telavancin, Tigecycline, Aminoglycosides, Carbapenems, Ceftazidime, Cefepime, Ceftobiprole, Ceftolozane/tazobactam, Fluoroquinolones, Piperacillin/tazobactam, Ticarcillin/clavulanic acid, Streptogramins, Daptomycin, Amikacin, Gentamicin, Kanamycin, Neomycin, Netilmicin, Tobramycin, Paromomycin, Streptomycin, Spectinomycin, Geldanamycin, Herbimycin, Rifaximin, Ertapenem, Doripenem, Imipenem/Cilastin, Meropenem, Cefadroxil, Cefazolin, Cephradine, Cephapirin, Cephalothin, and Cefalexin

Exemplary antimicrobial agents include Amikacin, Gentamicin, Kanamycin, Neomycin, Netilmicin, Tobramycin, Paromomycin, Streptomycin, Spectinomycin (Bs), Ansamycins, Geldanamycin, Herbimycin, Rifaximin, Carbacephem, Loracarbef, Carbapenems, Ertapenem, Doripenem, Imipenem/Cilastatin, Meropenem, Cefaclor, Cefoxitin, Cefotetan, Cefamandole, Cefmetazole, Cefonicid, Loracarbef, Cefprozil, Cefuroxime, Cephalosporins, Cefixime, Cefdinir, Cefditoren, Cefoperazone, Cefotaxime, Cefpodoxime, Ceftazidime, Ceftibuten, Ceftizoxime, Moxalactam, Ceftriaxone, Cephalosporins, Cefepime, Ceftaroline fosamil, Ceftobiprole, Glycopeptides, Teicoplanin, Vancomycin, Telavancin, Dalbavancin, Oritavancin, Lincosamides (Bs), Clindamycin, Lincomycin, Lipopeptide, Daptomycin, Macrolides (Bs), Azithromycin, Clarithromycin, Erythromycin, Roxithromycin, Telithromycin, Spiramycin, Fidaxomicin, Monobactams, Aztreonam, Nitrofurans, Furazolidone, Nitrofurantoin, Oxazolidinones (Bs), Linezolid, Posizolid, Radezolid, Torezolid, Penicillins, Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Temocillin, Ticarcillin, Polypeptides, Bacitracin, Colistin, Polymyxin B, Quinolones/Fluoroquinolones, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, Temafloxacin, Sulfonamides (Bs), Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole) (TMP-SMX), Sulfonamidochrysoidine (archaic), Tetracyclines (Bs), Demeclocycline, Doxycycline, Metacycline, Minocycline, Oxytetracycline, Tetracycline, Drugs against mycobacteria, Clofazimine, Dapsone, Capreomycin, Cycloserine, Ethambutol (Bs), Ethionamide, Isoniazid, Pyrazinamide, Rifampicin, Rifabutin, Rifapentine, Streptomycin, Arsphenamine, Chloramphenicol (Bs), Fosfomycin, Fusidic acid, Metronidazole, Mupirocin, Platensimycin, Quinupristin/Dalfopristin, Thiamphenicol, Tigecycline (Bs), Tinidazole, and Trimethoprim (Bs).

Exemplary antifungal agents include antifungal drugs in four main classes including polyenes, azoles, allylamines and echinocandins. Azoles are some of the most commonly used antifungals. They interfere with an enzyme that is important for creating the fungal cell membrane. Because of this, the cell membrane becomes unstable and can leak, eventually leading to cell death. There are two subgroups of azole antifungals: imidazoles and triazoles.

Exemplary imidazole antifungals include ketoconazole, clotrimazole, and miconazole. Exemplary triazoles include fluconazole, itraconazole, posaconazole, and voriconazole. Polyenes kill fungal cells by making the fungal cell wall more porous, which makes the fungal cell prone to bursting. Exemplary polyene antifungals include amphotericin B, nystatin, and allylamines. Echinocandins are a newer type of antifungal drug. They inhibit an enzyme that's involved in the making of the fungal cell wall. Exemplary echinocandins include anidulafungin, caspofungin, and micafungin. Other types of antifungal medications include flucytosine, which is an antifungal that prevents the fungal cell from making nucleic acids and proteins. Because of this, the cell can no longer grow and thrive. Flucytosine can be used to treat systemic infections with Candida or Cryptococcus species. Another type of antifungal medications includes griseofulvin, which works to prevent the fungal cell from dividing to produce more cells.

It is to be understood that the above lists are not exhaustive and that each antimicrobial agent in the lists may be more effective against certain bacteria and/or fungi. One of skill will readily be able to identify exemplary known antimicrobials and the bacteria and/or fungi they are effective against.

### V. Exemplary Antibiofilm Agents

Antibiofilm agents according to the present disclosure include any agents that degrade, depolymerize, and/or disrupt the various components of the microbial biofilm matrix. It is to be understood that the basic concepts of the present disclosure described herein are not limited by antibiofilm agent type.

Exemplary antibiofilm agents for use in the methods described herein include enzymes that degrade, e.g., extracellular DNA, polysaccharides and proteins of the biofilm matrix. Exemplary enzymes include nucleases, such as DNase I, polysaccharide hydrolases, dispersin B and proteases, and other enzymes known to those of skill in the art.

Further exemplary antibiofilm agents suitable for the methods disclosed herein comprise antimicrobial peptides that disrupt biofilms. Exemplary agents include nisin and LL-37.

Additionally, exemplary antibiofilm agents suitable for the methods disclosed herein include natural compounds that inhibit biofilm formation. Exemplary agents include curcumin and tea tree oil.

### VI. Methods of Therapeutic Treatment

The present disclosure describes methods of therapeutically treating a subject, such as an individual, such as a patient, such as a mammal, such as a human, in need thereof for a bacterial and/or fungal infection. The nanoparticles described herein are administered to an individual and treat a bacterial and/or fungal biofilm within the individual. The nanoparticles release an antimicrobial agent within the bacterial and/or fungal biofilm thereby treating the individual for the bacterial and/or fungal infection. The term “treating” or “treatment” is intended to include, but is not limited to, the prevention of the growth of a bacterial and/or fungal infection in a subject or inhibition or reduction in the growth of a pre-existing bacterial and/or fungal infection in a subject. In certain embodiments, the bacteria and/or fungi are sensitive to the antimicrobial agent so as to either kill the bacteria and or fungi, slow or inhibit the growth of the bacteria and/or fungi or prevent their replication. The nanoparticles also include an antibiofilm agent such as a nuclease. The nuclease degrades extracellular DNA associated with a bacterial and/or fungal biofilm, so as to reduce, disrupt or eliminate the bacterial and/or fungal biofilm or otherwise slow or inhibit growth of the bacterial and/or fungal biofilm.

In accordance with certain examples, nanoparticles as described herein can be incorporated into pharmaceutical compositions suitable for administration. Such pharmaceutical compositions typically comprise the nanoparticles disclosed herein and a pharmaceutically acceptable carrier. As used herein the term “pharmaceutically acceptable carrier” is intended to include any and all solvents, dispersion media, coatings, antimicrobial agents, antibacterial and antifungal agents, isotonic and absorption delaying agents, wound dressings, and the like, compatible with pharmaceutical administration. The use of such media and agents for pharmaceutically active substances is well known in the art. Except insofar as any conventional media or agent is incompatible with the nanoparticles or active agent, use thereof in the compositions is contemplated. Supplementary active compounds can also be incorporated into the compositions.

In accordance with certain examples, a pharmaceutical composition of the invention is formulated to be compatible with its intended route of administration. Such pharmaceutical compositions may be administered by inhalation, transdermally, topically, orally, rectally, transmucosally, intestinally, parenterally, intramuscularly, subcutaneously, intravenously or other suitable methods that will be readily selected by the person of ordinary skill in the art, given the benefit of this disclosure. For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

In accordance with other examples, pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMPHOR EL™ (BASF, Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

In accordance with other examples, sterile injectable solutions can be prepared by incorporating the nanoparticles in the required amount in an appropriate solvent with one or a combination of ingredients enumerated above, as required, followed by filtered sterilization. Generally, dispersions are prepared by incorporating the nanoparticles into a sterile vehicle which contains a basic dispersion medium and the required other ingredients from those enumerated above. In the case of sterile powders for the preparation of sterile injectable solutions, methods of preparation can be vacuum drying and freeze-drying which yields a powder of the nanoparticles plus any additional desired ingredient from a previously sterile-filtered solution thereof.

Oral compositions generally include an inert diluent or an edible carrier. Nanoparticles as described herein can be enclosed in gelatin capsules or compressed into tablets. For the purpose of oral therapeutic administration, the nanoparticles can be incorporated with excipients and used in the form of tablets, troches, or capsules. Oral compositions including the nanoparticles can also be prepared using a fluid carrier for use as a mouthwash, wherein the nanoparticles in the fluid carrier is applied orally and swished and expectorated or swallowed. Pharmaceutically compatible binding agents, and/or adjuvant materials can be included as part of the composition including the nanoparticles. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In at least certain examples, the nanoparticles are prepared with carriers that will protect the nanoparticles against degradation or rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In accordance with other examples, methods of treating a bacterial and/or fungal infection within a subject are disclosed. In one example, a method involves contacting a biofilm associated with a bacterial and/or fungal infection with nanoparticles including an antimicrobial and an antibiofilm agent such as a nuclease as described herein. Methods of treating or preventing a bacterial and/or fungal infection can be performed in vitro (e.g., by treating an in vitro or ex vivo surface having a bacterial biofilm thereon with nanoparticles as described herein) or, alternatively, in vivo (e.g., by administering the nanoparticles to a subject in need thereof). As used herein, a subject is intended to include both human and non-human mammals. Examples of non-human mammals include, but are not limited to, non-human primates, horses, cows, goats, sheep, dogs, cats, mice, rats, hamsters, guinea pigs and the like.

The present invention provides for both prophylactic and therapeutic methods of treating a subject for a bacterial and/or fungal infection. In one aspect, the invention provides a method for preventing a bacterial and/or fungal infection in a subject by administering to the subject the nanoparticles as described herein. Administration of prophylactic nanoparticles can occur prior to the manifestation of symptoms of a bacterial and/or fungal infection, such that the bacterial and/or fungal infection is prevented or, alternatively, delayed in its progression.

One embodiment of the present disclosure involves a method of treating a bacterial and/or fungal infection which includes the step of administering a therapeutically and/or prophylactically effective amount of nanoparticles as described herein to a subject. As defined herein, a therapeutically and/or prophylactically effective amount of nanoparticles (i.e., an effective dosage) ranges from about 0.001 to 30 mg/kg body weight, from about 0.01 to 25 mg/kg body weight, from about 0.1 to 20 mg/kg body weight, from about 1 to 10 mg/kg, from about 2 to 9 mg/kg, from about 3 to 8 mg/kg, from about 4 to 7 mg/kg, or from about 5 to 6 mg/kg body weight. The skilled artisan will appreciate that certain factors may influence the dosage required to effectively treat a subject, including but not limited to the severity of the bacterial and/or fungal infection, previous treatments, the general health and/or age of the subject, and other diseases or conditions present. Treatment of a subject with a therapeutically and/or prophylactically effective amount of nanoparticles can include a single treatment or can include a series of treatments. It will also be appreciated that the effective dosage of nanoparticles for treatment may increase or decrease over the course of a particular treatment.

### VII. Kits

In accordance with certain other examples, kits for treating bacterial and/or fungal infections in a subject are provided. A “kit” is a collection of parts forming the kit. In one example, the kit may include nanoparticles as described herein including an antimicrobial agent in a vial. The kit may include a pharmaceutically acceptable carrier in a vial. The kit may include nanoparticles in a pharmaceutically acceptable carrier in a vial. In an additional example, the kit may also include instructions for treating bacterial and/or fungal infections in a subject by administering the nanoparticles. In some examples, the kit may also comprise, e.g., a buffering agent, a preservative, or a protein stabilizing agent. In some examples, the kit may include an apparatus for administering the nanoparticles such as a syringe or other apparatus. Other suitable components for including in the kit will be selected by the person of ordinary skill in the art, given the benefit of this disclosure.

The following examples are set forth as being representative of the present disclosure. These examples are not to be construed as limiting the scope of the present disclosure as these and other equivalent embodiments will be apparent in view of the present disclosure, figures and accompanying claims.

### EXAMPLES

### Example I

### Initial Synthesis and Characterization of R-GNPs

The pH-responsive copolymer, methoxy PEG-benzoic-imine-branched polyethyleneimine (mPEG-b-i-bPEI) was synthesized as previously reported. See Nayeleh Deirram, Changhe Zhang, Sarah S. Kermaniyan, Angus P. R. Johnston, and Georgina K. Such. pH-Responsive Polymer Nanoparticles for Drug Delivery. Macromolecular Rapid Communications, 40(10):1-23, 2019 hereby incorproated by reference in its entirety. The successful synthesis of mPEG-b-i-bPEI was verified with Fourier-transform infrared spectroscopy and nuclear magnetic resonance spectroscopy.

Briefly, the functionalized GNPs were synthesized through a three-step synthesis (see FIG. 1, panel b). First, gelatin nanoparticles (“GNPs”) were prepared via a two-step desolvation method using glutaraldehyde as the crosslinker. See Yingying Wang and Anita Shukla. Bacteria-responsive biopolymer-coated nanoparticles for biofilm penetration and eradication. Biomaterials Science, pages 2831-2843, 2022 hereby incorporated by reference in its entirety.

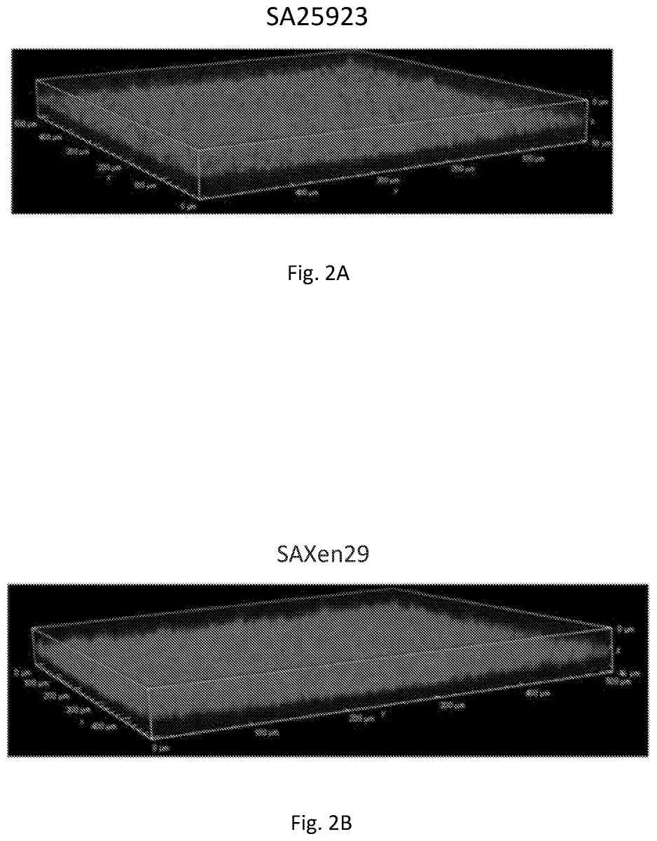

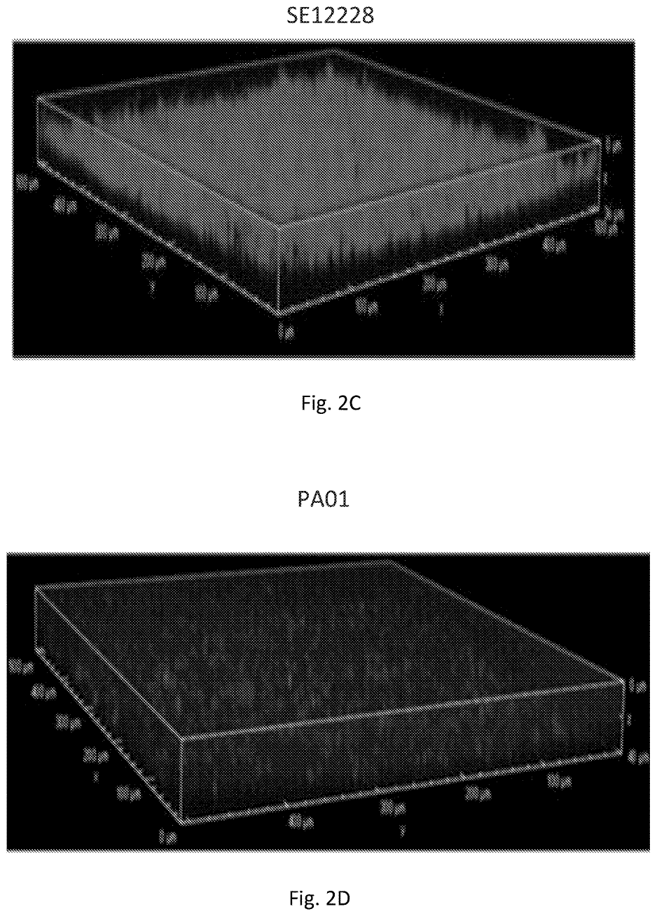

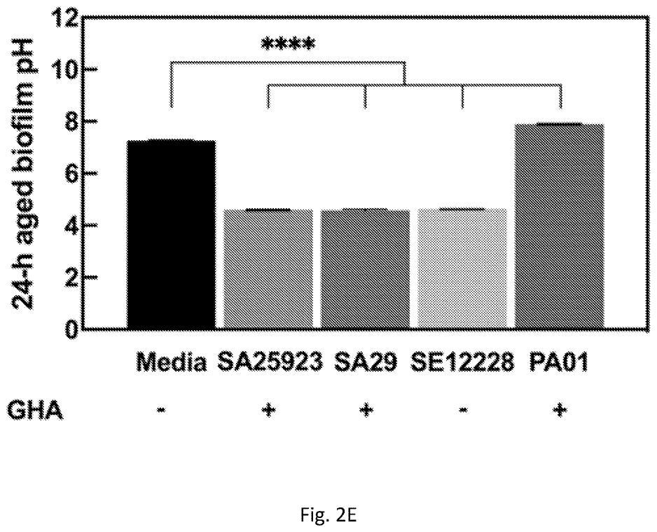

Then, AgNP-loaded GNPs (Ag-GNPs) were prepared by adding a solution of silver nitrate (AgNO₃) to the GNP suspension, followed by a sodium borohydride (NaBH₄) mediated reduction yielding AgNP formation within the GNPs. Finally, the Ag-GNPs were coated with DNase I and the pH-responsive copolymer via a two-step coupling reaction using 1-ethyl-3-(3-dimethylamino) propyl carbodiimide hydrochloride (EDC) and N-hydroxysuccinimide (NHS), yielding the complete responsive NP formulation (R-GNP). The morphology and size of the nanoparticles (“NPs”) at each step were assessed using transmission electron microscopy (TEM) and dynamic light scattering (DLS), respectively (FIG. 1, panels c-f). Inductively coupled plasma—optical emission spectrometry (ICP-OES) was used to quantify the amount of Ag in the GNPs, and AgNP composition was confirmed with energy-dispersive X-ray spectroscopy (EDX). The pH of the biofilm environment was measured using a pH probe, and the gelatinase activity of the biofilm was confirmed via a gelatin hydrolysis test, as previously reported (FIG. 2E). See Thomas Edison E. dela Cruz and Jeremy Martin O. Torres. Gelatin Hydrolysis Test Protocol. American Society for Microbiology, pages 1-10, 2012 hereby incorporated by reference in its entirety. The minimal biofilm eradication concentration to eradicate 80% of biofilm biomass (MBEC₈₀) was examined with crystal violet (CV) assays against Staphylococcus aureus 25923, S. epidermidis 12228, and Pseudomonas aeruginosa PA01. Finally, the cytotoxicity of the NPs was monitored via a hemolysis assay against 5% (v/v) human red blood cells (RBCs).

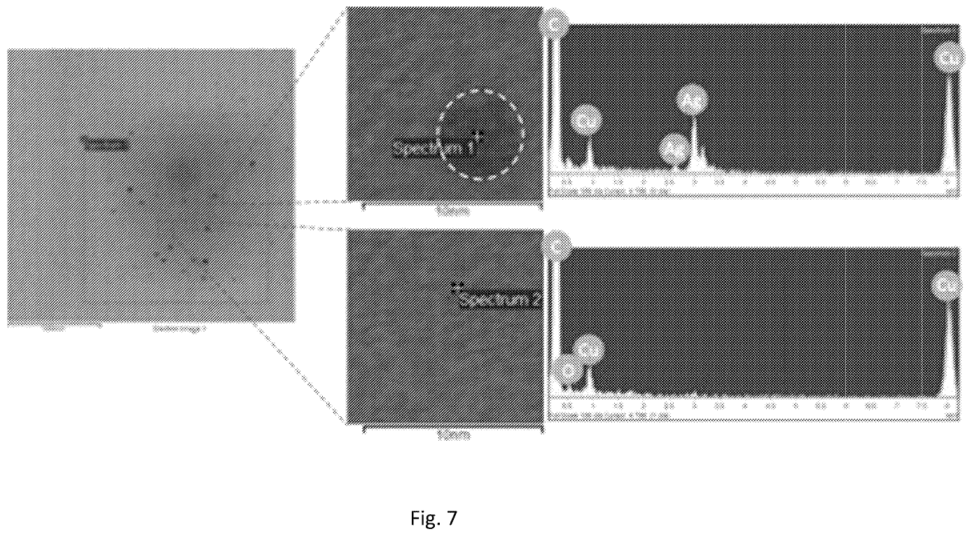

Nanoparticles were designed and engineered that exhibit pH- and bacterial enzyme-triggered drug release, resulting in antibiofilm efficacy. TEM imaging (see FIG. 1, panels c-e) and EDX analysis (FIG. 7) confirmed that AgNPs were successfully loaded into the GNPs. DLS showed an increase in NP hydrodynamic diameter with AgNP loading and DNase I functionalization, and further increase upon the addition of the pH-responsive polymer (see FIG. 1, panel f).

### Example II

### Additional Synthesis and Characterization of the R-GNPs

eDNA plays a key role in maintaining the structural stability of bacterial and/or fungal biofilms, with acidic conditions frequently observed in biofilms at infected wound sites. See Chen, Y., Zhao, Q., Han, J., Lan, X., Che, J., Chen, M., Liang, X. J., Ma, X. Dual Drug Loaded pH-sensitive Micelles for Efficient Bacterial Infection Treatment. Pharmaceutical Research, 2022, 39, 1165-1180; Gao, W., Vecchio, D., Li, J., Zhu, J., Zhang, Q., Fu, V., Li, J., Thamphiwatana, S., Lu, D., Zhang, L. Hydrogel containing nanoparticle-stabilized liposomes for topical antimicrobial delivery. ACS Nano, 2014, 8, 2900-2907; Schmid-Wendtner, M. H., Korting, H. C. The pH of the Skin Surface and Its Impact on the Barrier Function. Skin Pharmacology and Physiology, 2006, 19, 296-302; Wang, J. et al. PH-Switchable Antimicrobial Nanofiber Networks of Hydrogel Eradicate Biofilm and Rescue Stalled Healing in Chronic Wounds. ACS Nano, 2019, 13, 11686-11697. Various studies demonstrated the potential usage of employing DNase as an antibiofilm agent to eradicate bacterial and/or fungal biofilms. It is hypothesized that eDNA degradation would effectively loosen the biofilm matrix, enhancing NP penetration. Thus, R-GNP was developed for dual stimuli-responsive delivery, which incorporates AgNP as an alternative to antibiotics and is functionalized with DNase for biofilm disruption. The NPs are shielded by a pH-degradable co-polymer, designed to expose DNase in the acidic biofilm environment.

The presence of extracellular DNA (eDNA) in mature biofilms was first validated via confocal microscopy imaging. The results indicated that all bacteria of interest secrete eDNA during their biofilm stage (FIGS. 2A-2D). Within the tested biofilms, eDNA was stained with 7-hydroxy-9H-(1,3-dichloro-9,9-dimethylacridin-2-one) (DDAO), and the bacteria were stained with Hoechst stain. The DDAO stained the DNA fragments red, while the Hoechst stain colored the bacterial nuclei blue. Each sample was visualized under a confocal microscope, confirming the presence of eDNA in mature biofilms.

pH and gelatinases are responsive elements in the presently disclosed NP system. The addition of a pH-responsive copolymer enables the protection of DNase from external stimuli, allowing it to be exposed only until it encounters the biofilm matrix. The production of bacterial and/or fungal gelatinases triggers the degradation of the GNP core, releasing the encapsulated antimicrobial agents (i.e., AgNPs). Thus, pH levels and gelatinase production across different bacterial species was investigated. It was found that S. aureus and S. epidermidis produced acidic environments, while P. aeruginosa exhibited a basic environment during their biofilm stages (FIG. 2E).

Additionally, S. aureus and P. aeruginosa produced gelatinases during their biofilm stage, in contrast to S. epidermidis, which did not. Building on the understanding of these biofilm-specific responses, the R-GNPs were synthesized and validated for their design and functions.

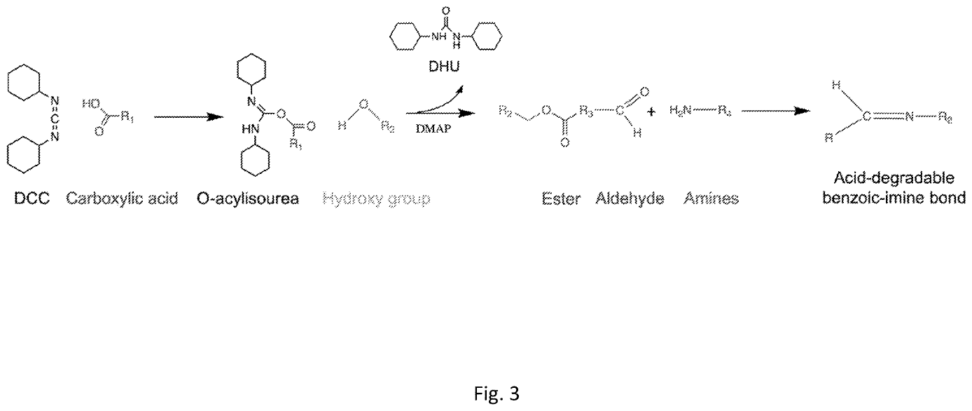

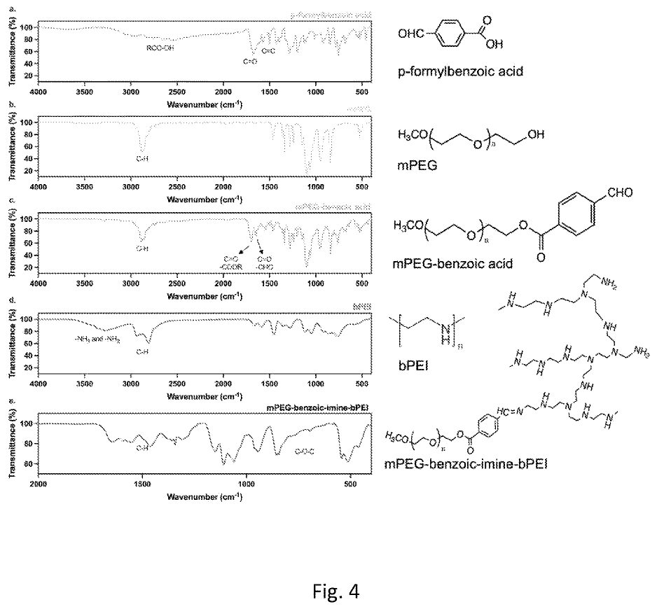

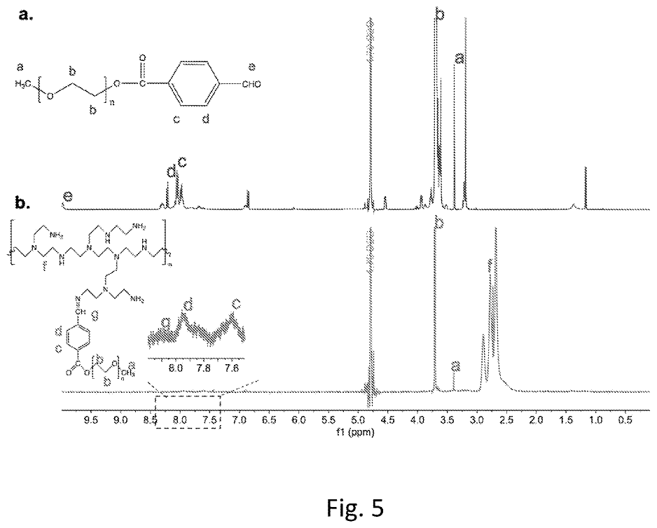

R-GNPs were developed to leverage the acidic milieu of biofilms and bacterial gelatinases for targeted therapeutic action. To synthesize the pH-responsive coating for R-GNP, PEG-benzoic-imine-polyethylenimine (PEG-b-i-bPEI) was first prepared (FIG. 3). This was achieved by initially generating PEG-benzoic acid through a reaction between the hydroxyl groups of PEG and the carboxyl groups of p-formylbenzoic acid. See Deirram, N., Zhang, C., Kermaniyan, S. S., Johnston, A. P., Such, G. K. pH-Responsive Polymer Nanoparticles for Drug Delivery. Macromolecular Rapid Communications, 2019, 40, 1-23; Park, W., Park, S. J., Shin, H., Na, K. Acidic Tumor pH-Responsive Nanophotomedicine for Targeted Photodynamic Cancer Therapy. Journal of Nanomaterials, 2016, 5, 1-8. Subsequently, benzoic-imine linkages were formed via a Schiff base reaction, involving the primary amine groups of PEI and the aldehyde groups of PEG-benzoic acid. The mPEG-benzoic-imine-bPEI (mPEG-b-i-bPEI) with benzoic-imine linkages was synthesized with the preparation of mPEG-benzoic acid (FIG. 3). This initial step involved a reaction between the hydroxyl group of mPEG and the carboxyl groups from p-formylbenzoic acid. The reactants and products at each step were characterized by FTIR and NMR (FIG. 4 and FIG. 5). Specifically, the FTIR spectrum of p-formylbenzoic acid (FIG. 4, panel a) showed a distinctive absorption band at 1677 cm-1, attributable to the C~O stretching vibration from aldehyde and carboxylic acid groups. Following the conjugation with mPEG, the FTIR spectrum of the resultant mPEG-benzoic acid (FIG. 4, panels b and c) exhibited absorption bands at 1097 cm-1 and within the range of 2883-3100 cm-1, corresponding to the C—O—C and C—H stretching vibrations of mPEG, respectively. The emergence of C═O stretching vibrations at 1697 cm-1 and 1646 cm-1 confirmed the successful conjugation of mPEG to p-formylbenzoic acid. This was further corroborated by NMR spectroscopy (FIG. 5, panel a), which revealed the characteristic proton peaks of mPEG-benzoic acid. Subsequent synthesis of mPEG-b-i-bPEI was achieved via a Schiff base reaction between the primary amine groups of bPEI and the aldehyde groups of mPEG-benzoic acid. Comparison of the FTIR spectrum of bPEI (FIG. 4, panel d) with that of mPEG-b-i-bPEI (FIG. 4, panel e) revealed the retention of the characteristic 1097 cm-1 C—O—C stretching vibration peak from mPEG-benzoic acid. Furthermore, the NMR spectrum of mPEG-b-i-bPEI (FIG. 5, panel b) indicated the disappearance of the aldehyde group's proton signal at δ 9.9 ppm and the emergence of the HC═N proton signal of the benzoic-imine linkage at δ 8.02 ppm, thereby confirming the successful conjugation of bPEI with mPEG-benzoic acid through the formation of benzoic-imine bonds.

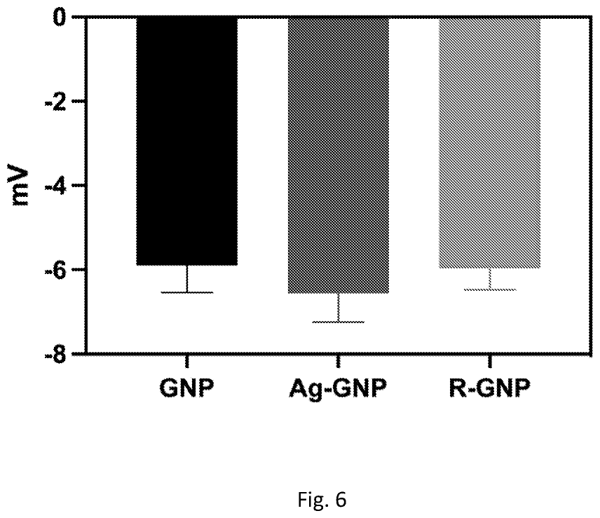

As described in Example I, GNPs were prepared employing a two-step desolvation method (See Wang, Y., Shukla, A. Bacteria-responsive biopolymer-coated nanoparticles for biofilm penetration and eradication. Biomaterials Science, 2022, 2831-2843; Coester, C. J., Langer, K., Von Briesen, H., Kreuter, J. Gelatin nanoparticles by two step desolvation-A new preparation method, surface modifications and cell uptake. Journal of Microencapsulation, 2000, 17, 187-193) and were subsequently loaded with AgNPs through a sodium borohydride (NaBH₄)-mediated reduction reaction (FIG. 1, panel b). The R-GNPs were then fabricated using the carbodiimide crosslinker chemistry (NHS/EDC) coupling reaction. During this process, DNase and mPEG-b-i-bPEI, both containing amine groups, were successfully conjugated onto the surface of the Ag-GNPs. Transmission electron microscopy (TEM) observations and dynamic size scattering (DLS) analysis confirmed that the NPs exhibited a well-defined spherical morphology and a uniform particle size. TEM images suggested that the loaded AgNPs appeared as black dots either on top or within the GNP core (FIG. 1, panels c and e). The hydrodynamic size of GNPs was around 177.2±5.6 nm. After loading with AgNPs, the size increased to 227.6±2.1 nm. The coating of DNase and the responsive co-polymer further increased particle size to 372.3±2.2 nm. DLS measurements demonstrated an increase in particle sizes, suggesting successful loading of AgNPs and the coating with DNase and the pH-responsive polymer (FIG. 1, panel f). ζ-potential measurements, taken with particles dispersed in 1×PBS (pH 7.40), showed the particles remain neutral throughout the synthesis. Specifically, the measurements were-5.89±0.64 mV for GNP, −6.57±0.68 mV for Ag-GNP, and −5.96±0.52 mV for R-GNP (FIG. 6). Energy-dispersive X-ray spectroscopy (EDX) analysis further confirmed the loading of AgNPs throughout the synthesis (FIG. 7), which revealed the presence of a highly intense absorption peak at 3 keV, a typical characteristic of nano-crystalline Ag. See Mavani, K., Shah, M. Synthesis of Silver Nanoparticles by using Sodium Borohydride as a Reducing Agent. International Journal of Engineering Research & Technology, 2013, 2, 1-8; Krishnaraj, C., Jagan, E. G., Rajasekar, S., Selvakumar, P., Kalaichelvan, P. T., Mohan, N. Synthesis of silver nanoparticles using Acalypha indica leaf extracts and its antibacterial activity against water borne pathogens. Colloids and Surfaces B: Biointerfaces, 2010, 76, 50-56. Additionally, a characteristic surface plasmon resonance (SPR) absorption peak appeared at 420 nm in the UV-vis spectra of Ag-GNP solution confirming the successful synthesis of AgNPs. Inductively coupled plasma optical emission spectroscopy (ICP-OES) analysis suggested 40±2 μg/mL and 14±7 μg/mL Ag+ were presented in Ag-GNPs and R-GNPs, respectively (FIG. 2E).

Furthermore, the amount of DNase coated was confirmed and quantified via enzyme-linked immunosorbent assay (ELISA). Enzyme-linked immunosorbent assay (ELISA) suggested that 5.48±0.31% (w/w) DNase I was conjugated onto the NP core.

### Example III

### The Stimuli-Responsiveness of the NPs

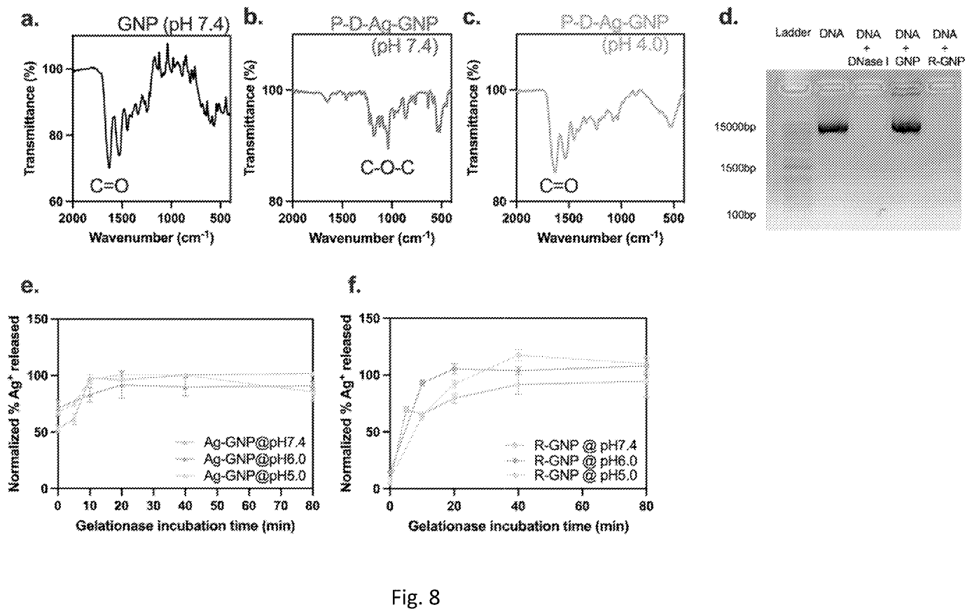

With the NP formulations successfully synthesized, the responsiveness of each NP element was tested. To evaluate the pH-responsiveness of R-GNPs, NPs were incubated in 1×PBS (pH 4.0) overnight, followed by freeze-drying. A control group of NPs at pH 7.4 with the same condition was included. Subsequent FTIR analysis of the resulting powder unveiled that the characteristic peak corresponding to the C—O—C stretching vibration of the pH-responsive polymer was absent, indicating a potential degradation of the polymer under acidic conditions (FIG. 8, panels a-c). Plasmid DNA is highly effective for validating DNase activity due to its well-defined structure, allowing for consistent interactions. The enzymatic effects can be quantified via gel electrophoresis, providing clear evidence of enzymatic digestion. Thus, to investigate the potential enzymatic activity of the R-GNP, an overnight incubation of the R-GNPs with plasmid DNA was conducted in a 1×PBS solution at pH 4.0. The results were then analyzed using agarose gel electrophoresis with a 1% agarose gel. The control group with plasmid DNA and DNase exhibited significant DNA degradation, confirming the nuclease activity of DNase. The plasmid DNA incubated with blank nanoparticles (i.e., GNPs) showed no signs of degradation. The plasmid DNA exposed to R-GNPs displayed clear degradation, demonstrating the preserved enzymatic activity of DNase when conjugated onto the R-GNPs (FIG. 8, panels d).

The release of AgNP in response to gelatinase was examined over 80 minutes incubation period at 37 °C, with 100 units (U) of gelatinases under various pH conditions (i.e., pH 5.0-7.4). The release of Ag+ was monitored via ICP-OES (FIG. 8, panels e and f). ICP-OES is utilized for detecting and quantifying trace amounts of metals, such as silver ions (Ag+), by measuring the light they emit at specific wavelengths. See Pimpimon Anekthirakun, Apichat Imyim, Microchemical Journal, 2019, 145, 470-475; ASTM-E3171-21a, 2021, Designation: E3171-21a, DOI: 10.1520/E3171-21A. The analysis revealed a gradual release of Ag+ from both Ag-GNPs and R-GNPs formulations, with 100% release achieved in 80 minutes under physiological pH conditions (i.e., pH 7.40), indicative of an enzyme-responsive release profile. It was observed that upon introducing gelatinases to the system, Ag-GNP responded rapidly with immediate release of Ag+, whereas R-GNPs showed a slower response. According to one aspect, the copolymer acts as a protective shell for the inner layers, thereby enhancing the stimuli-responsiveness of the NP system. Further investigation of Ag+ release under acidic pH conditions (i.e., pH 6.0 and 5.0) showed that Ag-GNP exhibited a release profile identical to its behavior at pH 7.4. In contrast, R-GNP demonstrated a more rapid release under acidic conditions compared to its behavior at pH 7.4, thereby providing antimicrobial efficacy, as sustained release of silver ions is known to enhance antimicrobial activity against a range of pathogens. Together, the results demonstrate that the NP system's design uses pH and enzyme responsiveness to optimize antimicrobial delivery.

### Example IV

### FTIR Confirms the Conjugation of mPEG-b-i-bPEI Copolymer to GNP

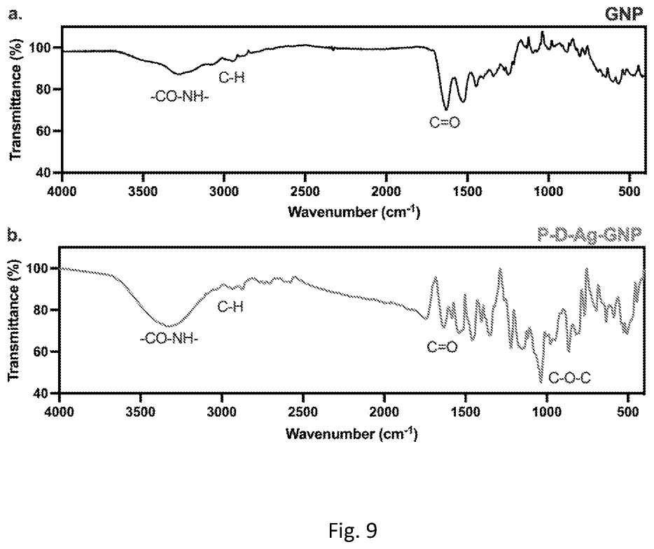

The FTIR spectra of synthesized NP formulation confirms successful conjugation of the mPEG-b-i-bPEI copolymer onto the GNP core (FIG. 9). In the GNP spectrum (FIG. 9, panel a), the absorption band at 3354 cm⁻¹ corresponds to the stretching vibrations of both N—H and O—H bonds. This is indicative of the presence of amine and hydroxyl groups in the structure, which is expected given that gelatin is a protein with amide linkages. See Nezhad-Mokhtari, P., Arsalani, N., Ghorbani, M., Hamishehkar, H. Development of biocompatible fluorescent gelatin nanocarriers for cell imaging and anticancer drug targeting. Journal of Materials Science, 2018, 53, 10679-10691. The band at 2940 cm⁻¹ can be attributed to the stretching vibrations of C—H bonds, commonly found in organic compounds with alkyl groups. (Id.) Notably, a pronounced peak at 1450 cm⁻¹ is associated with the aldimine linkage (CH═N). This linkage results from the reaction between the aldehyde group of glutaraldehyde, used as a cross-linking agent, and the amino group in the gelatin backbone. See Azimi, B., Nourpanah, P., Rabiee, M., Arbab, S. Producing gelatin nanoparticles as delivery system for bovine serum albumin. Iranian Biomedical Journal, 2013, 18, 34-40. The characteristic peak at 1097 cm⁻¹ indicated C—O—C stretching vibration of mPEG-benzoic acid, which confirmed the conjugation of the copolymer to the GNP core.

### Example V

### R-GNP Demonstrated Improved Inhibitory Effects Against Planktonic S. aureus

The impact of R-GNP on the growth of planktonic cells was assessed using optical density (OD) measurements and colony-forming unit (CFU) counting. The literature reports a wide range of minimum inhibitory concentration (MIC) and minimum bactericidal concentration (MBC) values for AgNPs. Specifically, MIC values for S. aureus have been found to vary between 1.14 and 128 μg/mL, with MBC values ranging from 10 to 60 μg/mL. For S. epidermidis, MIC values range from 0.76 to 94 μg/mL, and MBC values from 10 to 188 μg/mL. For P. aeruginosa, MIC values have been reported to range from 1.406 to 5.625 μg/mL, with MBC values between 2.813 and 5.625 μg/mL (Table1). See Swolana, D., K,epa, M., Idzik, D., Dziedzic, A., Kaba la-Dzik, A., Wasik, T. J., Wojtyczka, R. D. The antibacterial effect of silver nanoparticles on Staphylococcus epidermidis strains with different biofilm-forming ability. Nanomaterials, 2020, 10, 1-12; Sheikholeslami, S., Mousavi, S. E., Ahmadi Ashtiani, H. R., Hosseini Doust, S. R., Rezayat, S. M. Antibacterial activity of silver nanoparticles and their combination with zataria multiflora essential oil and methanol extract. Jundishapur Journal of Microbiology, 2016, 9; Wang, Y., Wang, Q. M., Feng, W., Yuan, Q., Yao, P., Sun, F. J., Xia, P. Y. Antibacterial activity and mechanism of moxifloxacin nanoparticles against drug-resistant Pseudomonas aeruginosa. Yaoxue Xuebao, 2020, 55, 2460-2465; Ambi, A., Parikh, N., Vera, C., Burns, K., Montano, N., Sciorra, L., Epstein, J., Zeng, D., Traba, C. Anti-infection silver nanoparticle immobilized biomaterials facilitated by argon plasma grafting technology. Biofouling, 2018, 34, 273-286; Dai, T., Wang, C., Wang, Y., Xu, W., Hu, J., Cheng, Y. A Nanocomposite Hydrogel 46 with Potent and Broad-Spectrum Antibacterial Activity. ACS Applied Materials and Interfaces, 2018, 10, 15163-15173; Bartel, M., Markowska, K., Strawski, M., Wolska, K., Mazur, M. Silver-decorated gel-shell nanobeads: Physicochemical characterization and evaluation of antibacterial properties. Beilstein Journal of Nanotechnology, 2020, 11, 620-630; El-Faham, A., Al-Rasheed, H. H., Sholkamy, E. N., Osman, S. M., Alothman, Z. A. Simple approaches for the synthesis of AgNPs in solution and solid phase using modified methoxypolyethylene glycol and evaluation of their antimicrobial activity. International Journal of Nanomedicine, 2020, 15, 2353-2362.

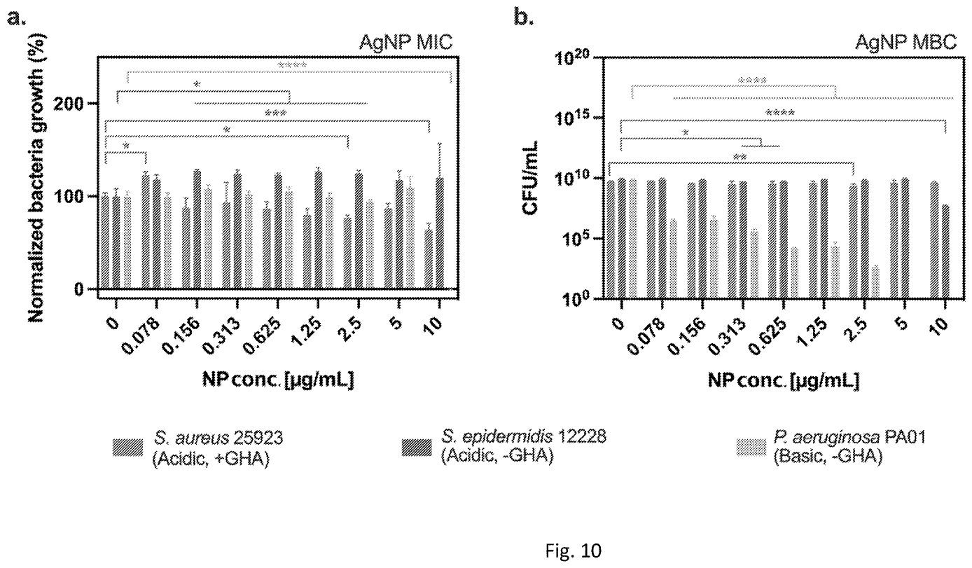

Additionally, the MIC and MBC were screened using commercially available AgNPs and observed that AgNPs were effective only in inhibiting and killing P. aeruginosa at a concentration of 10 μg/mL; they did not demonstrate inhibitory or bactericidal effects against S. aureus and S. epidermidis within the tested concentration range (i.e., 0-10 μg/mL) (FIG. 10, panels a and b, and Table1). AgNPs were tested against bacteria biofilm at different concentrations. No significant biofilm inhibitory and eradication efficacy was observed across all tested strains. It was hypothesized that the biofilm barrier prevents the penetration of AgNPs which results in compromised antibiofilm ability. Yuan et al. proposed that the bactericidal effects of AgNPs are mediated through their reactivity with various bacterial biomolecules, such as proteins, sugars, DNA, and enzymes. See Yuan, Y. G., Peng, Q. L., Gurunathan, S. Effects of silver nanoparticles on multiple drug-resistant strains of Staphylococcus aureus and Pseudomonas aeruginosa from mastitis-infected goats: An alternative approach for antimicrobial therapy. International Journal of Molecular Sciences, 2017, 18. This interaction results in genetic mutations and structural alterations to the bacterial cell wall, membrane, and proteins, ultimately inhibiting bacterial growth and cell death. The biofilm EPS acts as a physical barrier to prevent penetration which eventually blocks the interaction between AgNPs and secreted bacterial biomolecules. According to the present disclosure, embodiments are provided using biogenic AgNPs with heightened biofilm penetration activity to address bacterial infections.

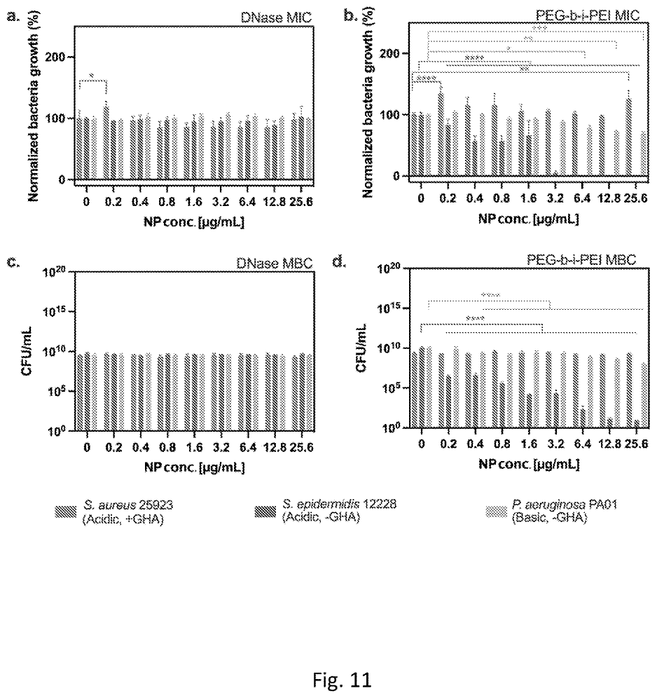

Additionally, the MIC and MBC for DNase and co-polymer were also tested. DNase did not show antimicrobial effects against all tested strains (FIG. 11, panels a and c). The co-polymer did not inhibit antimicrobial or bactericidal effect against S. aureus and P. aerugionsa; however, it demonstrated inhibitory (MIC₈₀) and bactericidal (MBC₈₀) effects against S. epidermidis planktonic cells at 3.2 μg/mL and 25.6 μg/mL, respectively (FIG. 11 panels b and d). Thus, the initial objective was to identify the MIC and MBC for NP formulations. The disparity in efficacy observed across different bacterial strains underscores the need for targeted nanoparticle treatments, aligning with the goal of developing more effective antibacterial strategies. The antimicrobial activity of DNase I and the responsive copolymer (mPEG-b-i-bPEI) was also evaluated in this study utilizing S. aureus 25923, S. epidermidis 12228 and P. aeruginosa PA01. The MICs and MBCs of both agents were determined (FIG. 11, panels a-d). These results indicate that DNase I did not exhibit any antimicrobial activity against all three tested bacterial strains (FIG. 11, panels a and c). Interestingly, mPEG-b-i-bPEI demonstrated a notable inhibitory effect against S. epidermidis 12228 at a concentration as low as 3.2 μg/mL (FIG. 11, panel b). Additionally, regarding bactericidal activity, mPEG-b-i-bPEI did not exhibit any bactericidal effects against S. aureus 25923 and P. aeruginosa PA01 (FIG. 11, panel d). mPEG-b-i-bPEI was able to reduce the viable cell count of S. epidermidis 12228 by 3 log CFU/mL at a concentration of 0.8 μg/mL (FIG. 11, panels b and d).

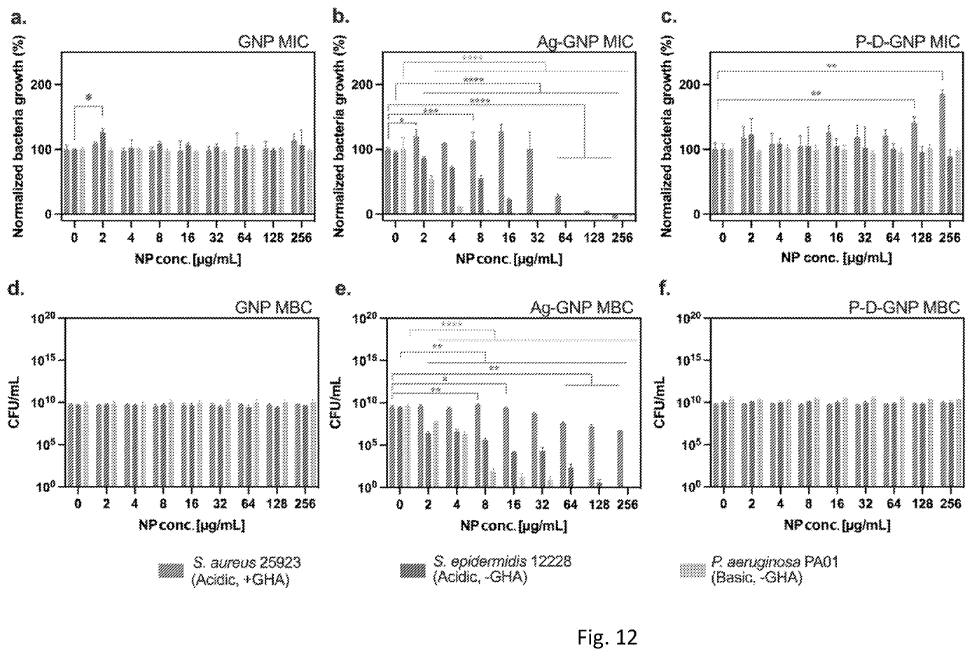

According to one aspect, bacteria of interest were exposed to various concentrations of NP formulations to investigate their inhibitory and bactericidal concentrations against planktonic cells. More specifically, planktonic bacteria were treated with NP formulations for 16 to 18 hours, after which the MIC was quantified by OD measurement, expressed as a percentage (%) of normalized bacterial growth in comparison to untreated controls. The MBC resulting from overnight NP formulation treatment was quantified via CFU counting and expressed as CFU per mL (CFU/mL). Blank NPs (i.e., GNP), as well as NPs containing only the antibiofilm agent (i.e., P-D-GNP), did not exhibit bacterial inhibitory (FIG. 12, panels a and c) or bactericidal effects (FIG. 12, panels d and f) against all tested strains (i.e., S. aureus 25923, S. epidermidis 12228 and P. aeruginosa PA01) (Table1). Unlike the outcomes observed with the other two tested strains, GNPs equipped solely with the antimicrobial agent (i.e., Ag-GNP) neither inhibited bacterial growth nor exhibited bactericidal effects against S. aureus within the tested concentrations (0.2 to 265 μg/mL). The minimal inhibitory concentrations required to inhibit 80% (MIC₈₀) of bacterial growth were 32 μg/mL for S. epidermidis and 4 μg/mL for P. aeruginosa, as shown in FIG. 12, panel b and Table1. Furthermore, Ag-GNPs were capable of killing more than 80% of the bacteria (MBC₈₀) at concentrations of 128 μg/mL for S. epidermidis and 32 μg/mL for P. aeruginosa (FIG. 12, panel e and Table1). These MIC and MBC results indicate that S. aureus is more resistant to Ag+ ions and/or AgNPs compared to S. epidermidis and P. aeruginosa. Additionally, the ability of P. aeruginosa to secrete gelatinases may enhance the release of Ag+ and/or AgNPs from the GNP shell, facilitating the inhibitory and bactericidal effects. Moreover, gram-negative bacteria, including P. aeruginosa, proved to be more susceptible to Ag+ ions and/or AgNPs, likely because their cell wall composition does not provide as effective a barrier against NP penetration as seen in Gram-positive bacteria like S. aureus and S. epidermidis. See Swolana, D., K,epa, M., Idzik, D., Dziedzic, A., Kaba la-Dzik, A., Wasik, T. J., Wojtyczka, R. D. The antibacterial effect of silver nanoparticles on Staphylococcus epidermidis strains with different biofilm-forming ability. Nanomaterials, 2020, 10, 1-12; Liao, C., Li, Y., Tjong, S. C. Bactericidal and cytotoxic properties of silver nanoparticles. International Journal of Molecular Sciences, 2019, 20. The MIC findings underscore the need for refining nanoparticle-based antimicrobial strategies, specifically by incorporating antibiofilm agents hypothesized to enhance the clearance of S. aureus for optimal effectiveness.

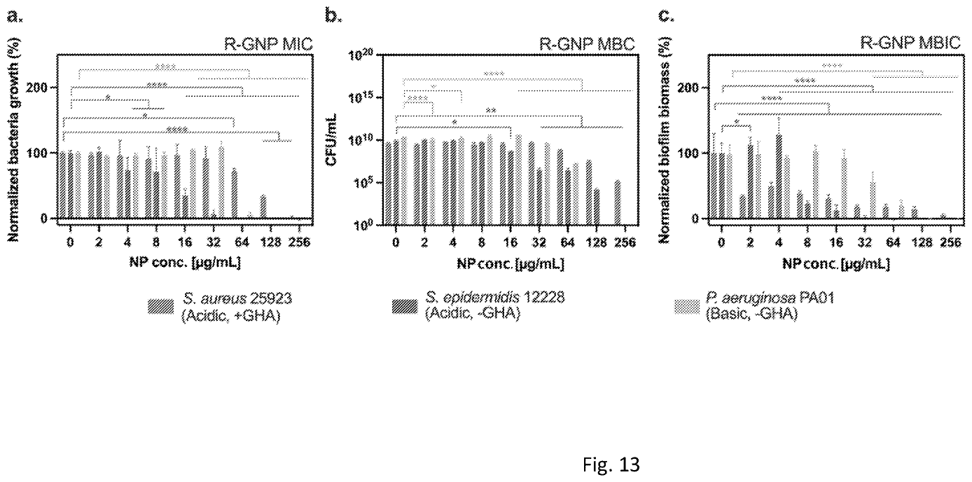

Numerous studies have highlighted DNase ability to inhibit biofilm formation and detach pre-formed biofilms, with some research also demonstrating its antimicrobial activity against Staphylococci. See, Kaplan, J. B. Therapeutic potential of biofilm-dispersing enzymes. 2009, 32, 545-554; Kaplan, J. B., Lovetri, K, Cardona, S. T., Madhyastha, S., Sadovskaya, I., Jabbouri, S., Izano, E. A. Recombinant human DNase I decreases biofilm and increases antimicrobial susceptibility in staphylococci. Journal of Antibiotics, 2012, 65, 73-77; Thomas, V. C., Thurlow, L. R., Boyle, D., Hancock, L. E. Regulation of autolysisdependent extracellular DNA release by Enterococcus faecalis extracellular proteases influences biofilm development. Journal of Bacteriology, 2008, 190, 5690-5698. When equipped with the antibiofilm agent DNase, the responsive NP formulation (i.e., R-GNP) demonstrated enhanced bacterial inhibitory efficacy against S. aureus (p<0.0001), with a MIC₈₀ of 256 μg/mL, compared to that observed with Ag-GNP. Additionally, no changes in MIC₈₀ for S. epidermidis were noted, and a decrease in antimicrobial efficacy against P. aeruginosa was observed (FIG. 13, panels a and b and Table 1). The MIC results for R-GNP suggest that the alkaline environment of P. aeruginosa may facilitate the degradation of the pH-responsive copolymer (FIG. 2E), ultimately affecting the stability of GNP and the release of Ag+ ions and/or AgNPs. Thus, the initial MIC results indicate that DNase enhances the antimicrobial activity of AgNP against S. aureus. Accordingly, the present disclosure describes a dual stimuli-responsive delivery system combining antibiofilm and antibacterial agents as an effective strategy to combat bacterial biofilm infections. FIG. 12 demonstrated the results for evaluating the antibacterial effects of GNP, Ag-GNP, and P-D-GNP against S. aureus 25923, S. epidermidis 12228 and P. aeruginosa PA01 where a-c depict the minimal inhibitory concentration (MIC) of the NPs and d-f illustrate the minimal bactericidal concentration (MBC) of the NPs. No inhibitory and bactericidal effect of GNP against all three bacterial strains (FIG. 12, panels a and e). Once loaded with silver, Ag-GNP can inhibit the growth of S. aureus 25923 at 128 μg/mL, S. epidermidis 12228 at 8 μg/mL, and P. aeruginosa PA01 at 32 μg/mL (FIG. 12, panel b). However, Ag-GNP were not able to completely kill all S. aureus 25923 cells, it achieves a 2 log CFU reduction at 64 μg/mL (FIG. 12, panel e). Additionally, all S. epidermidis 12228 cells are eradicated at 64 μg/mL, and all P. aeruginosa PA01 cells at 128 μg/mL (FIG. 12, panel e). The coating of the responsive copolymer and DNase I (P-D-GNP) neither inhibit nor kills any of the three tested strains (FIG. 12, panels c and f). Accordingly, the present disclosure describes that Ag-GNP exhibits both inhibitory and bactericidal effects against the tested strains at specific concentrations, whereas GNP and P-D-GNP do not exhibit any inhibitory or bactericidal effects, highlighting Ag-GNP as an exemplary antibacterial agent.

TABLE 1
List of MIC and MBC values for NP formulations, including
laboratory test values using commercially available
AgNPs★ (SA = S. aureus 25923, SE =
S. epidermidis 12228, PA = P. aeruginosa PA01).

| | MIC₈₀ (μg/mL) | | | MBC₈₀ (μg/mL) | | |
|---|---|---|---|---|---|---|
| Sample | SA | SE | PA | SA | SE | PA |
| AgNP^{★} | — | — | 10 | — | — | 5 |
| GNP | — | — | — | — | — | — |
| P-D-GNP | — | — | — | — | — | — |
| Ag-GNP | — | 32 | 4 | — | 128 | 32 |
| R-GNP | 256 | 32 | 64 | — | 256 | 128 |

### Example VI

### R-GNP Demonstrated Strong Biofilm Eradication Efficacy Against S. aureus Biofilms

The literature reveals a broad spectrum of MBIC and MBEC values for AgNPs targeting biofilms of the pathogens of interests. Specifically, with notable variations depending on the synthesis method of the nanoparticles, MBIC values for S. aureus have been found to range from 4 to 400 μg/mL; (See Bartel, M.; Markowska, K.; Strawski, M.; Wolska, K.; Mazur, M. Silver-decorated gel-shell nanobeads: Physicochemical characterization and evaluation of antibacterial properties. Beilstein Journal of Nanotechnology 2020, 11, 620-630. Singh, P.; Kim, Y. J.; Wang, C.; Mathiyalagan, R.; El-Agamy Farh, M.; Yang, D. C. Biogenic silver and gold nanoparticles synthesized using red ginseng root extract, and their applications. Artificial Cells, Nanomedicine and Biotechnology 2016, 44, 811-816. Ramachandran, G.; Alharbi, N. S.; Chackaravarthy, G.; Kanisha Chelliah, C.; Rajivgandhi, G.; Maruthupandy, M.; Quero, F.; Natesan, M.; Li, W. J. Chitosan/silver nanocomposites enhanced the biofilm eradication in biofilm forming Gram positive S. aureus. Journal of King Saud University—Science 2023, 35, 102597; dos Santos, E. M. P.; Martins, C. C. B.; de Oliveira Santos, J. V.; da Silva, W. R. C.; Silva, S. B. C.; Pelagio-Flores, M. A.; Galembeck, A.; Cavalcanti, I. M. F. Silver nanoparticles-chitosan composites activity against resistant bacteria: tolerance and biofilm inhibition. Journal of Nanoparticle Research 2021, 23, 1-13; Thomas, R.; Soumya, K. R.; Mathew, J.; Radhakrishnan, E. K. Inhibitory effect of silver nanoparticle fabricated urinary catheter on colonization efficiency of Coagulase Negative Staphylococci. Journal of Photochemistry and Photobiology B: Biology 2015, 149, 68-77.) For S. epidermidis, MBIC values range from a remarkably low 0.01 μg/mL to 3 μg/mL; (See Bartel, M.; Markowska, K.; Strawski, M.; Wolska, K.; Mazur, M. Silver-decorated gel-shell nanobeads: Physicochemical characterization and evaluation of antibacterial properties. Beilstein Journal of Nanotechnology 2020, 11, 620-630; Kalishwaralal, K.; BarathManiKanth, S.; Pandian, S. R. K.; Deepak, V.; Gurunathan, S. Silver nanoparticles impede the biofilm formation by Pseudomonas aeruginosa and Staphylococcus epidermidis. Colloids and Surfaces B: Biointerfaces 2010, 79, 340-344; Swolana, D.; K,epa, M.; Kruszniewska-Rajs, C.; Wojtyczka, R. D. Antibiofilm Effect of Silver Nanoparticles in Changing the Biofilm-Related Gene Expression of Staphylococcus epidermidis. International Journal of Molecular Sciences 2022, 23.) And for P. aeruginosa, MBIC values have ranged from 8 to 50 μg/mL.80,84,85 In terms of MBEC values, for S. aureus, they vary significantly, from 50 to 1000 μg/mL, depending on the nanoparticle synthesis and formulation. (See Hamedi, S.; Shojaosadati, S. A.; Mohammadi, A. Evaluation of the catalytic, antibacterial and anti-biofilm activities of the Convolvulus arvensis extract functionalized silver nanoparticles. Journal of Photochemistry and Photobiology B: Biology 2017, 167, 36-44; Ansari, M. A.; Khan, H. M.; Khan, A. A.; Cameotra, S. S.; Alzohairy, M. A. Anti-biofilm efficacy of silver nanoparticles against MRSA and MRSE isolated from wounds in a tertiary care hospital. Indian Journal of Medical Microbiology 2015, 33, 101-109; Moyano, A. J.; Mas, C. R.; Colque, C. A.; Smania, A. M. Dealing with biofilms of Pseudomonas aeruginosa and Staphylococcus aureus: In vitro evaluation of a novel aerosol formulation of silver sulfadiazine. Burns 2020, 46, 128-135) Similarly, for P. aeruginosa, MBEC values are reported between 5 and 250 g/mL, (See Moyano, A. J.; Mas, C. R.; Colque, C. A.; Smania, A. M. Dealing with biofilms of Pseudomonas aeruginosa and Staphylococcus aureus: In vitro evaluation of a novel aerosol formulation of silver sulfadiazine. Burns 2020, 46, 128-135; Bjarnsholt, T.; Kirketerp-Møller, K.; Kristiansen, S.; Phipps, R.; Nielsen, A. K.; Jensen, P.; Høiby, N.; Givskov, M. Silver against Pseudomonas aeruginosa biofilms. Apmis 2007, 115, 921-928; Loo, C. Y.; Young, P. M.; Cavaliere, R.; Whitchurch, C. B.; Lee, W. H.; Rohanizadeh, R. Silver nanoparticles enhance Pseudomonas aeruginosa PAO1 biofilm detachment. Drug Development and Industrial Pharmacy 2014, 40, 719-729.) In the case of S. epidermidis, the MBEC values span from 0.05 to 50 μg/mL. (See Ansari, M. A.; Khan, H. M.; Khan, A. A.; Cameotra, S. S.; Alzohairy, M. A. Anti-biofilm efficacy of silver nanoparticles against MRSA and MRSE isolated from wounds in a tertiary care hospital. Indian Journal of Medical Microbiology 2015, 33, 101-109; Chaw, K. C.; Manimaran, M.; Tay, F. E. Role of silver ions in destabilization of intermolecular adhesion forces measured by atomic force microscopy in Staphylococcus epidermidis biofilms. Antimicrobial Agents and Chemotherapy 2005, 49, 4853-4859.) AgNPs are effective to manage bacterial biofilms.

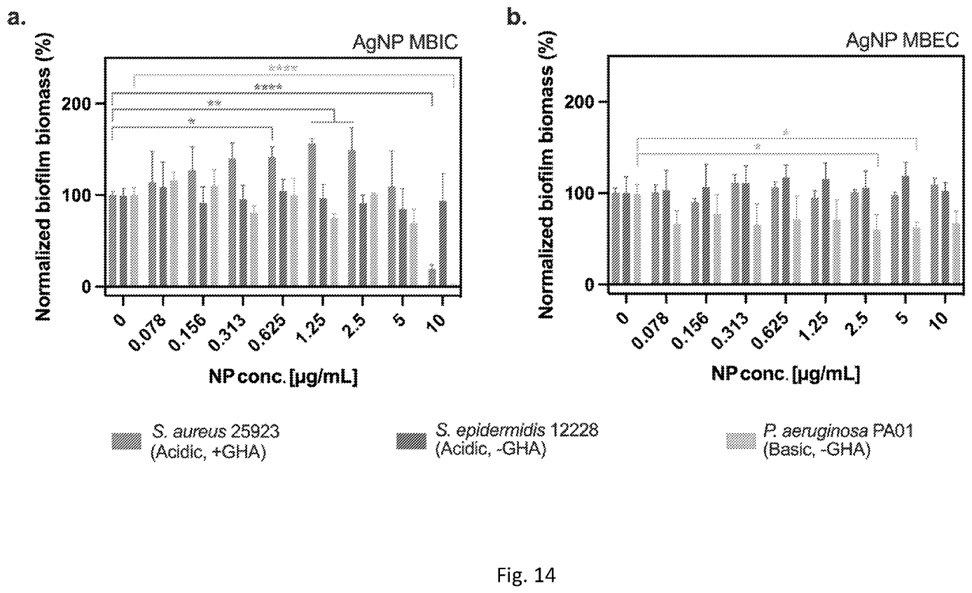

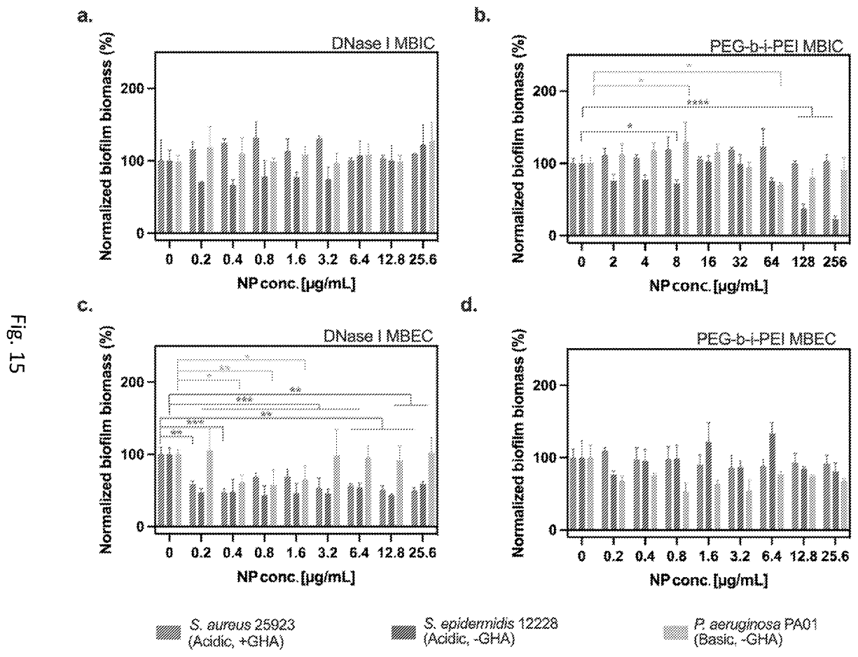

According to the present disclosure, crystal violet (CV) staining, scanning electron microscopy (SEM) imaging, and confocal microscopy were used to investigate changes in biofilm biomass and morphology upon treatment with R-GNP. Biofilm biomass was stained with 0.1% (v/v) CV and quantified in terms of MBIC and MBEC across various NP formulation concentrations. Before analyzing MBIC and MBEC in NP formulations, the inhibitory and bactericidal efficacy was evaluated using commercially available AgNPs. The results indicate that Ag-NPs inhibit more than 80% of S. aureus and P. aeruginosa biofilms at a concentration of 10 μg/mL, whereas such were not able to inhibit biofilm formation of S. epidermidis biofilm formation (FIG. 14, panel a, and Table 2). Additionally, AgNPs were not able to eradicate bacterial biofilm against all these bacteria within the tested concentration ranges (i.e., 0-10 μg/mL) (FIG. 14, panel b and Table 2).

FIG. 15 (Panels a-d) shows the antibiofilm efficacy of DNase I and a pH-responsive copolymer. Neither DNase I nor the pH-responsive copolymer showed biofilm inhibitory effects against any of the tested strains. However, DNase I demonstrated a biofilm eradication effect against all tested strains, indicating that DNase I is an effective antibiofilm agent.

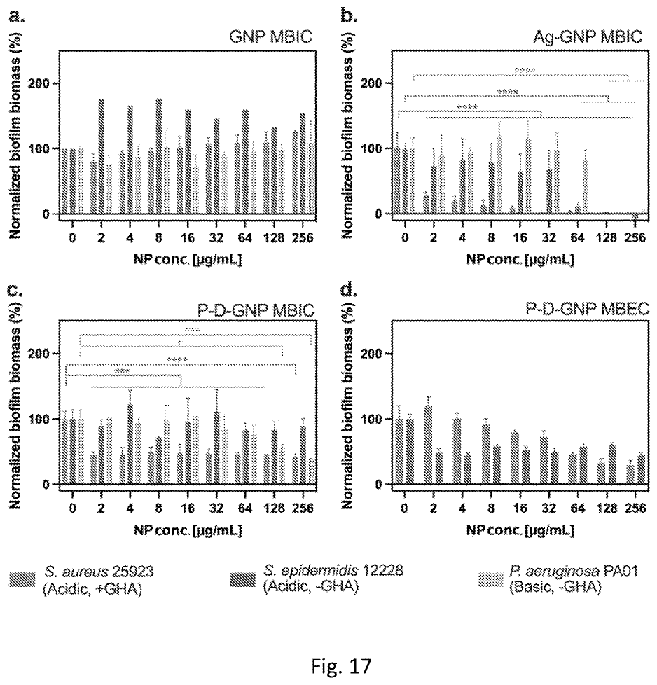

In the MBIC studies, NP formulations were added simultaneously with bacterial cells and cultured under static conditions to promote biofilm formation. After a 24-hour incubation and a subsequent washing step, the biofilm biomass was stained and assessed. The MBIC results show that GNPs and P-D-GNPs were not able to inhibit biofilm formation (FIG. 17, panels a and c, and Table 2). Ag-GNPs could inhibit 80% of biofilm formation (MBIC₈₀) at a concentration of 32 μg/mL for S. aureus, which increased to 64 and 128 μg/mL for S. epidermidis and P. aeruginosa, respectively (FIG. 17, panel b and Table 2). With the addition of an antibiofilm agent, the biofilm inhibitory efficacy remained the same for S. aureus and improved for P. aeruginosa to a concentration of 32 μg/mL, while remaining unchanged for S. epidermidis. Accordingly, the present disclosure describes significant biofilm inhibitory effects of RGNP, especially when combined with an antibiofilm agent, to provide a system for treating biofilm-associated bacterial infections.

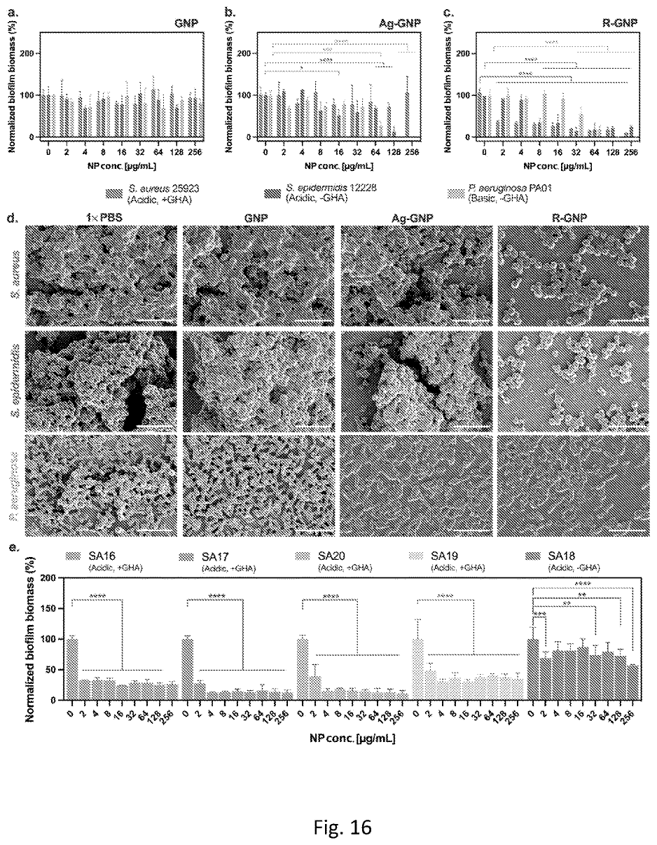

For the MBEC studies, pre-formed biofilm was treated with NP formulations for 24 hours, and the biofilm biomass remaining after washing was stained and evaluated. The MBEC studies showed no biofilm eradication effect of treatment with blank NP (i.e., GNP) and P-D-GNP for all tested bacterial strains (FIG. 16, panel a and FIG. 17, panel d and Table 2). GNP loaded with antimicrobial agent alone (i.e., Ag-GNPs) was able to eradicate 80% S. epidermidis and P. aeruginosa biofilm biomass (MBEC₈₀) at concentrations at 128 μg/mL, whereas no eradication was observed for S. aureus biofilm within the tested concentrations (FIG. 16, panel b and Table 2). When coated with the antibiofilm agents, the NP formulation demonstrated significant improvement (P<0.0001) to eradicate S. aureus biofilm with concentration at 32 μg/mL (FIG. 16, panel c and Table 2). Additionally, improved eradication against S. epidermidis and P. aeruginosa biofilm was also observed (FIG. 16, panel c and Table 2). Importantly, both S. aureus and S. epidermidis biofilm biomass decreased upon treatment with lower concentrations of R-GNPs (MBEC₈₀=34 μg/mL for both) compared to what was needed for P. aeruginosa biofilm (MBEC₈₀=64 μg/mL; FIG. 16, panel c) demonstrating that the acidic biofilm environment of S. aureus and S. epidermidis initiate the release of DNase.

The SEM images further confirmed the biofilm eradication results (FIG. 16, panel d and FIG. 19). Compared with the GNP and Ag-GNP-treated biofilms, R-GNP treatment led to a drastic disruption of the Staphylococcus biofilm structure. Overall, the responsive NP formulation demonstrated significant eradication of biofilms that exhibited acidic environments (i.e., S. aureus and S. epidermidis). At low R-GNP concentrations, the lack of gelatinase production by S. epidermidis appeared to reduce biofilm eradication efficacy compared to what was observed for S. aureus. Additionally, limited eradication of the basic P. aeruginosa biofilm was observed, despite gelatinase production, supporting the hypothesized mechanism of action of the R-GNPs.

Furthermore, the biofilm eradication efficacy of R-GNP against S. aureus clinical isolates obtained from Rhode Island Hospital was explored. Each isolate, derived from patients with tissue infections, presented a unique opportunity to assess the translational applicability of nanoparticles described herein. According to the present disclosure, R-GNP significantly eradicates biofilms from S. aureus clinical isolates (p<0.0001), demonstrating its potent antibiofilm properties (FIG. 16, panel e). Further, R-GNP exhibited a pronounced biofilm eradication ability against S. aureus clinical isolates known to produce gelatinases, compared to those isolates lacking gelatinase production (FIG. 16, panel e). This differential efficacy underscores the stimuli-responsiveness of the R-GNP formulation, where gelatinases modulate the activity of R-GNP by affecting its penetration into the biofilm matrix or by interacting with the NP components directly.

TABLE 2
List of MBIC and MBEC values for NP formulations,
including laboratory test values using commercially available
AgNPs^{★} (SA = S. aureus 25923, SE =
S. epidermidis 12228, PA = P. aeruginosa PA01).

| | MBIC₈₀ (μg/mL) | | | MBEC₈₀ (μg/mL) | | |
|---|---|---|---|---|---|---|
| Sample | SA | SE | PA | SA | SE | PA |
| AgNP^{★} | 10 | — | 10 | — | — | — |
| GNP | — | — | — | — | — | — |
| P-D-GNP | — | — | — | — | — | — |
| Ag-GNP | 32 | 64 | 128 | — | 128 | 128 |
| R-GNP | 32 | 32 | 128 | 32 | 32 | 64 |

### Example VII

### DNase Coating Enhanced NP Penetration into S. aureus Biofilms

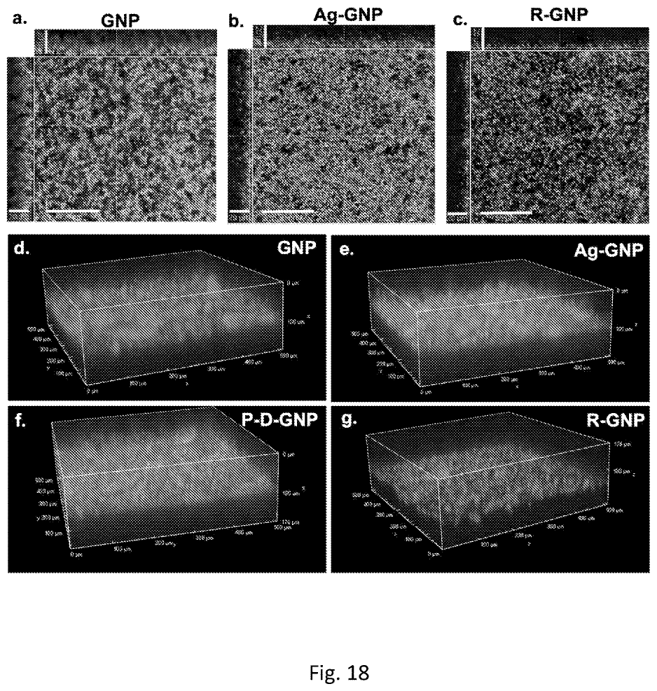

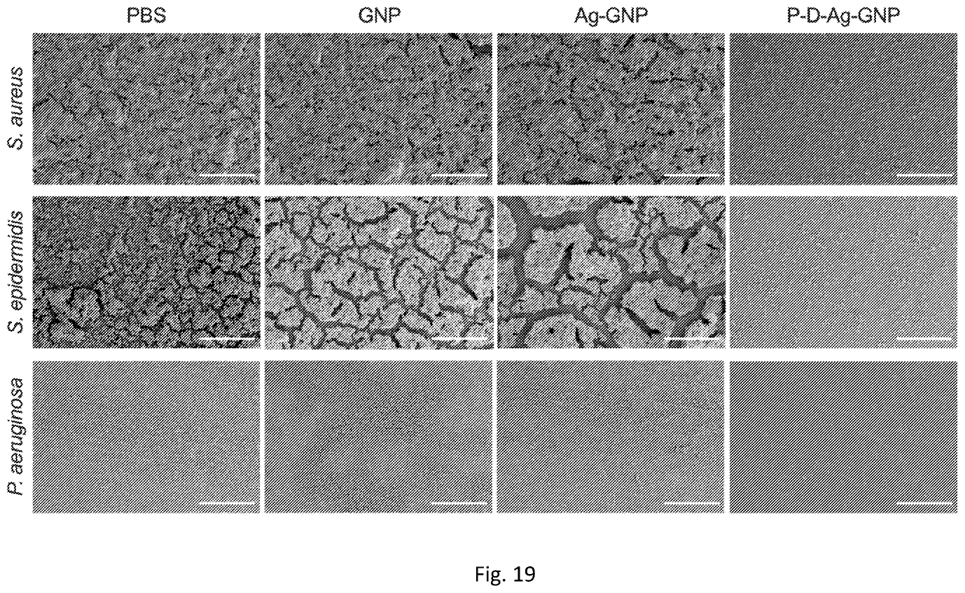

The efficacy of R-GNP in eradicating and penetrating S. aureus biofilms was further investigated. Opera Phonex high content screen system was utilized to assess the viability of S. aureus within biofilms following treatment with NP formulations, employing live/dead staining to distinguish between live and dead bacteria. The live/dead assay, which stains live bacteria green and dead bacteria red, revealed a significant decrease in the proportion of live bacteria within the biofilms treated with Ag-GNPs and further decreased treated with R-GNPs compared to controls (FIG. 18, panels a-c). The live/dead staining results demonstrate enhanced antibacterial efficacy of R-GNPs against S. aureus biofilms. The significant decrease in live bacteria within treated biofilms underscores the potential of DNase coating in eradicating biofilm integrity and enhancing nanoparticle-mediated killing.

Confocal laser scanning microscopy (CLSM) was employed to visualize the interaction between the DNase-coated NPs and the biofilms. The FilmTracer Ruby Stain was utilized to stain the biofilm matrix. The results demonstrated a significant disruption of the S. aureus biofilms upon treatment with NP formulations. CLSM images revealed that the biofilm's extracellular matrix, which typically provides structural integrity and protection to the bacterial community, was substantially degraded upon overnight treatment with RGNP.

This degradation facilitated the penetration of the nanoparticles deep into the biofilms (FIG. 18, panels d-g). The ability of DNase-coated NP to disrupt and penetrate S. aureus biofilms can be attributed to several factors. Firstly, the DNase coating degrades eDNA, which is a critical component of the biofilm matrix. eDNA contributes to the structural integrity of biofilms and mediates bacterial adhesion and aggregation. Its degradation leads to a weakened biofilm structure, making it more susceptible to penetration by therapeutic agents. Secondly, the release of AgNP from the core can be delivered directly into the biofilm, offering a targeted approach to treating biofilm-associated infections.

FIG. 19 displays lower magnification scanning electron microscope images of all tested strains compared to the images provided in FIG. 16, Panel d. A significant amount of biofilm eradication is observed in all images when treated with the R-GNP formulation compared to their respective controls.

### Example VIII

### In Vitro Cell Viability and Cytotoxicity Studies

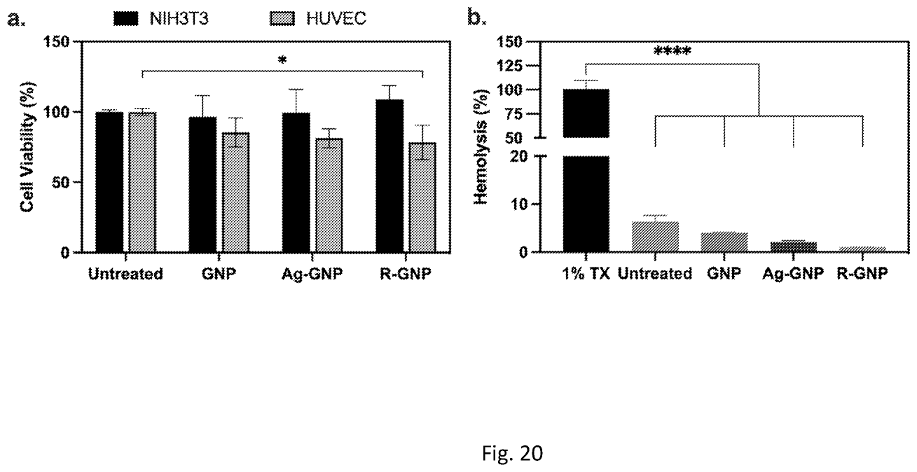

The toxicity towards mammalian cells was evaluated using NIH 3T3 fibroblasts, HUVECs, and human red blood cells. All nanoparticle (NP) formulations showed no cytotoxicity, with cell viability exceeding 80% across all groups compared to the untreated controls (FIG. 20, panel a). Additionally, all NP formulations exhibited no hemolytic effects against human red blood cells. The DNase and responsive polymer coating effectively reduced the hemolysis typically observed with Ag-GNPs. (FIG. 20, panel b)

### Example IX

### R-GNP Shows Strong Antibiofilm Activity Against S. aureus on Porcine Skin Ex Vivo

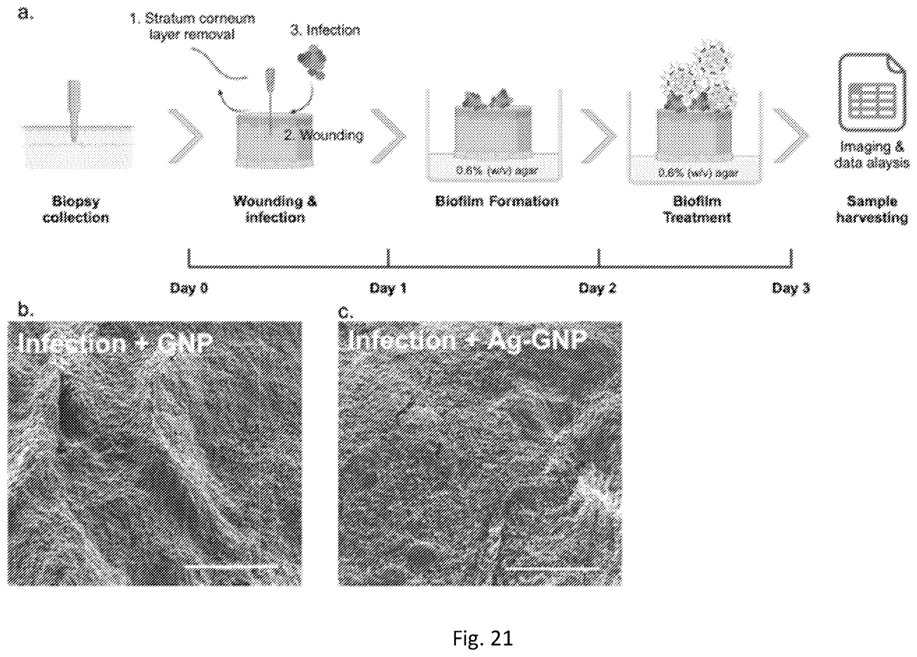

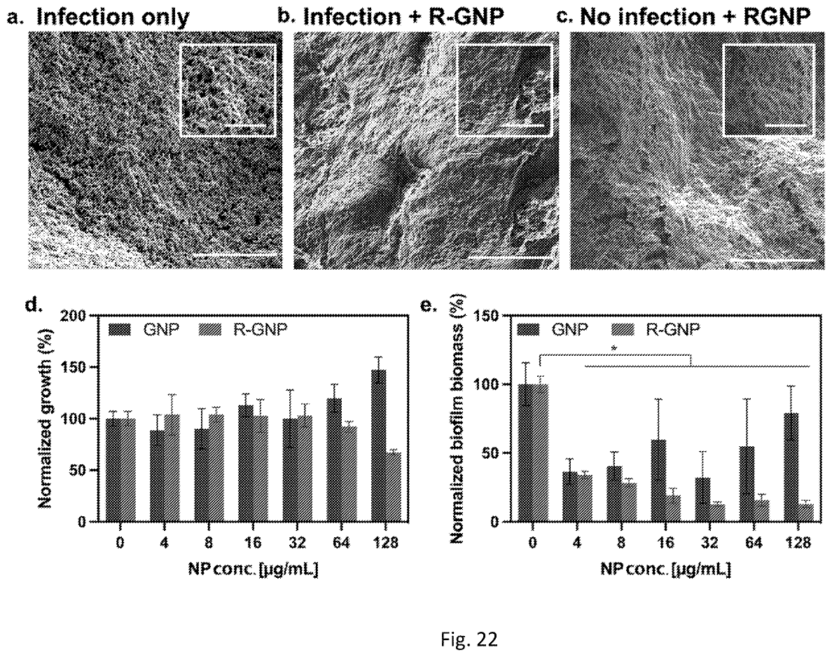

To study S. aureus biofilm infection on skin and the effects of treatment, porcine skin and S. aureus were utilized as a platform for establishing infection and monitoring treatment efficacy. FIG. 21, Panel a, presents a schematic of the approach used to utilize ex vivo porcine skin infections for investigating the biofilm eradication efficacy of R-GNP. Control wounds, left untreated, exhibited a thicker layer of microbe biofilm on the wound surface, as evidenced by SEM images (FIG. 22, panel a.); A negative control sample, which consisted of uninfected skin explants treated with R-GNP (FIG. 22, panel c), was also included. Additionally, infected wounds treated with GNP and Ag-GNPs did not exhibit a significant decrease in biofilm biomass compared to the control group (FIG. 21, panels b and c). Most notably, wounds treated with R-GNP demonstrated a reduction in biofilm biomass on the skin surface, suggesting that the R-GNP formulation is effective in eradicating biofilms from infected wounds (FIG. 22, panel b).

Additionally, based on the morphology of the microbes observed in the SEM and histology images, porcine skin exhibited a polymicrobial infection containing both fungal and bacterial cells. Consequently, MIC and MBIC assays were performed to investigate the antifungal efficacy of the R-GNP formulation against C. albicans SC5314 planktonic and biofilm samples (FIG. 22, panels d and e). Although no significant inhibition of C. albicans cells was observed (FIG. 22, panel d), the R-GNP formulation significantly inhibited the formation of C. albicans biofilms (FIG. 22, panel e), thereby demonstrating the formulation's potential in eradicating polymicrobial biofilms.

### Example X

### In Vivo Antibiofilm Efficacy

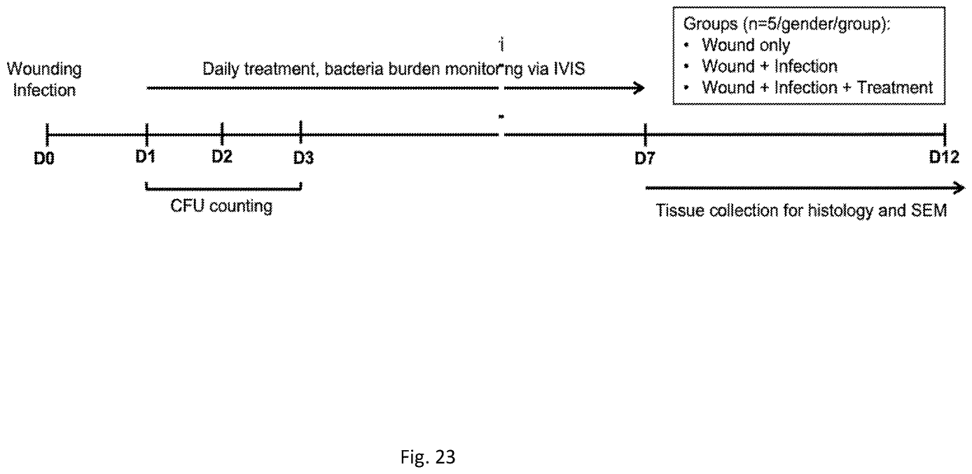

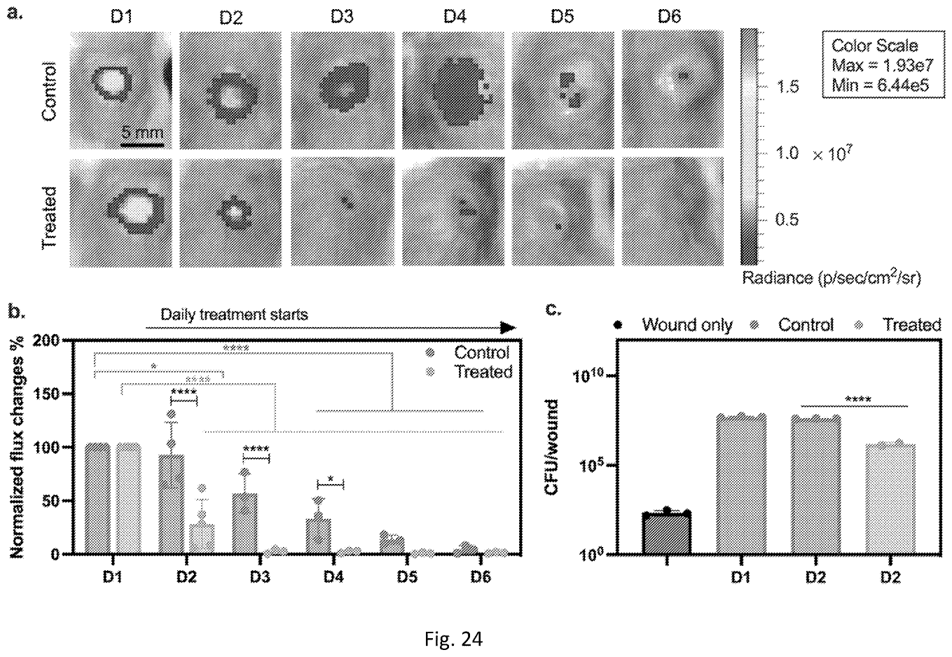

To facilitate the translation of R-GNP therapy, its efficacy against S. aureus in clinical isolates from infected tissues was explored using an in vivo murine excisional biofilm wound infection model. Balb/c WT mice (both male and female), aged 7-8 weeks, were acquired from Jackson Lab. Each mouse was wounded and then infected with bioluminescent S. aureus Xen29. The luminescence signal was monitored daily via IVIS 24 hours post-infection (Experiment layout was illustrated in FIG. 23). A 40 μg dose of R-GNP was topically administered via injection starting on day 1. Significant eradication of bacterial biofilm was observed 24 hours post-treatment, with continued reduction following subsequent treatments (FIG. 24, panel a). By quantifying the IVIS signal, the R-GNP formulation was found to eradicate 70% of the biofilm compared to control groups on Day 2 with decreasing bioluminescent signal following subsequent treatments (FIG. 24, panel b). Additionally, the cell forming unit (CFU) pre- and post-treatment was quantified, where significant CFU reduction was observed post-reatment (FIG. 24, panel c).

### Example XI

### Experimental Materials and Methods

Gelatin (type B, blood strength of ~225 g), glutaraldehyde (25% (w/w) in water), silver nitride (AgNO₃), sodium borohydride (NaBH₄), acetone, ethanol (200 proof, anhydrous), methanol, poly(ethylene glycol) methyl ether (mPEG, molecular weight (M.W.)=2 KDa), branched polyethyleneimine (bPEI) (M.W.=2 KDa), p-formylbenzoic acid (95%), Nhydroxysuccin-imide (NHS), N,N-dicyclohexylcarbodiimide (DCC, 99%), 4-dimethylaminopyridine (DMAP, 98%), deuterium oxide (D₂O), hydrochloric acid (1M), D-glucose, crystal violet (CV), carbonate bicarbonate and 10× Dulbecco's phosphate-buffered saline (PBS, pH 7.4) were purchased from Sigma-Aldrich (St Louis, MO). Tryptic soy broth (TSB) and agar were purchased from Thermo Scientific. Pooled human blood plasma was purchased from Innovative Research. Deionized water was produced from MiliQ synthesis (18 MΩ cm, Millipore Sigma, Billerica, MA). All chemicals were reagent grade and used as received. paraformaldehyde (Electron Microscopy Sciences, Hatfield, PA) ethanol (Greenfield Global, Brookfield, CT).

### Preparation of NPs Formulations

Synthesis of mPEG-b-i-bPEI Co-Polymer

The synthesis of mPEG-b-i-bPEI copolymer based on Schiff base reaction of mPEG-benzoic acid and bPEI. See Kaul, G., Amiji, M. Tumor-targeted gene delivery using poly(ethylene glycol)-modified gelatin nanoparticles: In vitro and in vivo studies. Pharmaceutical Research, 2005, 22, 951-961. The mPEG-benzoic acid was first synthesized by the DCC/DMAP-mediated esterification of mPEG with p-formylbenzoic acid. (Id.) p-formylbenzoic acid (4.73 g), mPEG (6 g), DCC (6.52 g) and DMAP (0.96 g) were dissolved in dry DCM (100 mL). The reaction was performed under stirring at room temperature for 24 hours. Once DCC was removed via filtration, the solution was concentrated by a rotary evaporator. Subsequently, the product was collected by precipitation from cold diethyl ether and washed with isopropanol. The product was dried under a vacuum overnight at room temperature. Subsequently, bPEI (100 mg), mPEG-CHO (20 mg) and DMAP (10 mg) were directly dissolved in DMSO (2.0 mL). The solution was stirred at RT for 72 hours and then dialyzed (Cellu Sep MWCO 3500) against 1×PBS at 4 °C for 24 h to remove DMSO and the product was collected by lyophilization.

### Preparation of GNP and Ag-GNP

GNPs were synthesized via a two-step desolvation technique, employing glutaraldehyde as the cross-linking agent. See Wang, Y., Shukla, A. Bacteria-responsive biopolymer-coated nanoparticles for biofilm penetration and eradication. Biomaterials Science, 2022, 2831-2843. Initially, gelatin was solubilized in Milli-Q water to achieve a 50 mg/mL concentration at 50 °C. During the first desolvation step, an equal volume of acetone was incrementally added to the gelatin solution under unvarying stirring at 360 rpm. This resulted in the precipitation of high molecular weight gelatin fractions. Subsequently, the supernatant, which contained the lower molecular weight gelatin, was removed. The precipitate was then resuspended in an equivalent volume of Milli-Q water at 50 °C. The solution's pH was adjusted to 3.0 using a 1M HCl solution. For the second desolvation step, acetone was added dropwise to the gelatin solution at a rate of 1 mL/min under continuous stirring, thus forming the GNPs. Cross-linking was initiated by the gradual addition of 0.12% (v/v) GA, and the reaction mixture was left to stir overnight at ambient temperature. The resultant GNPs were isolated by centrifugation, washed thrice with Milli-Q water, and conserved at 4 °C in Milli-Q water for subsequent applications.

AgNP-loaded GNPs (i.e., Ag-GNPs) were prepared by adding a solution of silver nitrate (AgNO₃) to the GNP suspension, followed by a sodium borohydride (NaBH₄) mediated reduction yielding AgNP formation within the GNPs. The mixture was then stirred continuously for 4 hours at room temperature. Ag-GNPs were collected by centrifugation and washed three times with Milli-Q water. The final products were stored at 4 °C in Milli-Q water for future use.

### Preparation of R-GNPs

To prepare the R-GNPs, Ag-GNPs were first resuspended in 1×PBS (pH 7.4). To activate the carboxylic acid groups, 0.4 mg of EDC and 11 mg of sulfo-NHS were first dissolved in 0.25 ml of MES buffer (pH 5.5). This mixture was then added to the Ag-GNP suspension and incubated at room temperature for 15 minutes. Subsequently, DNase solution (1 mg/mL in 1×PBS) was added to the nanoparticle solution, followed by stirring for 15 minutes at room temperature. Next, mPEG-b-i-bPEI copolymer (1 mg/mL in 1×PBS) was introduced, and the reaction was allowed to proceed under gentle stirring at 4 °C overnight. Post-reaction, the nanoparticles were dialyzed against 1×PBS using a 300 kDa dialysis membrane to remove unbound DNase and excess polymer, also conducted overnight at 4 °C, The R-GNPs were stored in 1×PBS (pH 7.4) at 4 °C.

### Characterization of NPs

### Characterization of NP Morphology

The size and surface charge of the NPs were examined by dynamic light scattering (DLS) and zeta (2) potential measurement using a Malvern Zetasizer Nano ZS (Zetasizer Nano ZS90, Malvern Instruments, UK, operating at a scattering angle of) 90°. The morphology of the NPs was characterized by transmission electron microscopy (TEM) imaging using a JEM-2100 electron microscope operating at an acceleration voltage of 200 kV. NP formulations in 1×PBS (pH 7.4) were prepared by drop-casting onto copper grids and dried overnight at room temperature in the dark.

### Confirmation of Silver Loading

Inductively coupled plasma-optical emission spectrometry (ICP-OES iCAP, 7400 DUO, Thermo Fisher Scientific, USA) was used to quantify the amount of Ag in the GNPs. Briefly, Ag-GNPs or R-GNPs were digested in 70% (v/v) nitric acid at 95 °C for 2 hours at a concentration of 1 mg/mL, where colourless solutions indicated complete digestions. Samples were then diluted with deionized water to a final 2% (v/v) nitric acid concentration. Radial plasma view configuration was used for measuring the ion intensity relative to an Ag standard (Inorganic Ventures, Christiansburg, VA). NP composition was confirmed with energy-dispersive X-ray spectroscopy (EDX) coupled with TEM.

Confirmation of Enzyme and pH-Responsive Co-Polymer Coating

The mPEG-b-i-bPEI conjugation was confirmed via FTIR. Briefly, 1 mg NPs were freezedried overnight and the spectrum of each sample was then obtained using a Bruker Alpha-P ATR FTIR. The amount of DNase coated on R-GNP was confirmed via ELISA following manufacturing protocol. The activity of DNase on R-GNPs was monitored by incubating with plasmid DNA (10 ng/μL) in R-GNPs suspension (200 μg/mL) for 10 minutes at 37 °C with orbital shaking at 100 rpm. The cleavage of plasmid DNA was analyzed via gel electrophoresis (1.5% (w/v) agarose gel stained with SYBR safe).

### Characterization of NP Stimuli-Responsiveness

The NP gelatinases-responsiveness was confirmed by incubating with 100 μg/mL collagenase IV (i.e., gelatinases) for various time points. At each timepoint, NPs were isolated via centrifugation using a 1 nm spin unit filter, spent media was collected and the release of silver was monitored via ICP-OES.

### Characterization of NP Stability

The stability of the particles was analysed by measuring the size and surface charge of the NPs under different pH values (from 4 to 8) in 1×PBS, and under different conditions including TSB and TSB supplemented with 1% (w/v) glucose (TSBG) and DMEM/F12.

### In Vitro Characterization of NP Antimicrobial and Antibiofilm Efficacy

### Bacteria Strains and Growth Media

S. aureus American Type Culture Collection (ATCC) 25923, S. auerus Xen29, P. aeruginosa PA01 and S. epidermidis ATCC12228 were routinely cultured in TSB broth (Thermo Scientific, USA, containing 17.0 g/L soytone, 3.0 g/L glucose, 2.5 g/L sodium chloride and 5.0 g/L dipotassium phosphate). Overnight cultures of all tested strains were prepared by inoculating bacterial glycerol stock into TSB followed by incubation at 37 °C with orbital shaking at 200 rpm for 16-18 h. The overnight culture was used to inoculate fresh nutrient rich medium at a ratio of 1:500 (v:v) and incubated at 37 °C with shaking at 200 rpm until reaching the exponential phase. The growth phase was monitored by measuring the optical density at 600 nm (OD₆₀₀) using a spectrophotometer until it reached a value of OD₆₀₀=0.1.

Bacteria biofilms were prepared by inoculating 1×10⁶ colony forming units per mL of culture (CFU/mL) exponential phase bacteria in TSBG. Biofilms were formed by static incubation at 37 °C for 24 h in tissue culture treated 96-wells plate coated with 20% (v/v) human blood plasma diluted in carbonate bicarbonate buffer (pH 8).

### Biofilm Eradication Assay

The efficacy of NP therapeutics on biofilm eradication was evaluated using CV and microbial cell viability assays. Various concentrations of NP therapeutics were applied to biofilms aged for 24 hours. Biofilm biomass was assessed through the CV assay. In brief, biofilms were first washed once with 1×PBS after the supernatant was discarded. They were then fixed with 100% methanol and stained with 0.1% (w/v) CV solution. Excess CV was decanted, and the biofilms were washed three times with 1×PBS. The stained biomass was solubilized in 100% methanol, and the OD of the CV was measured at 590 nm (OD₅₉₀), with higher readings indicative of increased biomass. The percentage of remaining biofilm biomass was determined using Equation (1). The concentration of therapeutic that eradicated the biofilm was also recorded as the MBEC.

$$Normalized viability ( \% ) = \frac{{OD}_{590 , sample} - {OD}_{590 , drug , NC}}{{OD}_{590 , PC} - {OD}_{590 , NC}} \times 100 \qquad ( 1 )$$

### Biofilm SEM Imaging

Before SEM imaging, biofilms were formed on silicon wafers under the same conditions as described above. The biofilm samples were treated with various NP formulations and control groups and gently washed three times with 1×PBS. The samples were then fixed with a 2% (v/v) glutaraldehyde, 2% (v/v) paraformaldehyde, in 1×PBS solution at 4 °C for 4 hours. After washing with 1×PBS, the samples were dehydrated by incubating with ascending ethanol (Greenfield Global, Brookfield, CT) series (50%, 60%, 70%, 80%, 90%, and 100% (v/v)) for 10 min each at room temperature. Samples were air-dried overnight at room temperature and gold-palladium was sputter-coated at 20 mA for 60 seconds with a sputter coater (Jeol JFC-1600). Samples were then imaged with a SEM (Quattro S, Thermo Fisher Scientific, USA) operated at 2 kV.

### Biofilm Confocal Imaging

Biofilms were cultured on silicon wafers, replicating the conditions outlined previously. Following a 24-hour exposure to the therapeutic compounds and controls at 37 °C, the treatments were discarded, and the biofilms were rinsed with 1×PBS. Subsequently, biofilms were stained utilizing LIVE/DEAD BacLight Bacterial Viability dyes (Thermo Fisher Scientific, USA) following the manufacturer's protocol. The stained biofilms were visualized using a 63×water immersion lens on the Opera Phenix Plus High-Content Screening System (PerkinElmer, Waltham, MA). For image analysis, Harmony high-content analysis software was employed. Image acquisition was executed at 0.5 μm steps to a maximal depth of 20 μm from the base, across six regions measuring 40,000 μm² within quadruplicate wells.

### In Vitro Cytotoxicity Studies

### Hemolysis Assay

The potential cytotoxicity of the therapeutics was tested using the hemolysis test. A PC (positive control) of 1% (v/v) triton X-100 (i.e., 100% lysis) and a NC (negative control) of 1×PBS (i.e., 0% lysis) are used. Released haemoglobin is measured at 540 nm (OD₅₄₀), and the percent (%) hemolysis is quantified using Equation (2).

$$Normalized viability ( \% ) = \frac{{OD}_{540 , Therapeutic} - {OD}_{540 , NC}}{{OD}_{540 , PC} - {OD}_{540 , NC}} \times 100 \qquad ( 2 )$$

### CCK-8 Assay

Fibroblast cell (NIH3T3) or endothelial cells (HUVEC) were seeded and cultured at the 5000 cells/well in 96-well tissue culture plates. Following 24-hours incubation, cells were treated with NP formulations (i.e., GNP, Ag-GNP and R-GNP) and experimental control (i.e., 1×PBS). Positive and negative controls of cells lacking treatment and media alone, respectively, were included as well. Following a 24-hour incubation, cells were washed with 1×PBS three times and media containing 10% (v/v) CCK-8 solution. After incubation for 2 hours at 37 °C supplied with 5% CO₂, the absorbance was measured at 450 nm and viability was calculated using Equation (3).

$$Normalized viability ( \% ) = \frac{{OD}_{450 , Therapeutic} - {OD}_{450 , NC}}{{OD}_{450 , PC} - {OD}_{450 , NC}} \times 100 \qquad ( 3 )$$

### Ex Vivo Antibiofilm Efficacy Studies

### Procine Skin Collection and Storage

Skin samples from Yucatan pigs weighing between 85 and 95 kg were collected for ex vivo skin infection experiments. All procedures followed strict aseptic techniques, performed by veterinarians. The samples came from pigs previously used in biomechanical studies, which had no systemic infections or medications. Collection occurred within one-hour post-mortem. Hairs were removed with a clipper and hair removal cream (Nair), followed by cleansing with water. The skin was then disinfected with 70% (v/v) ethanol, dried with sterile gauze, and exposed to UV light for 15 minutes. Using a surgical scalpel, we excised the skin into small pieces, approximately 10×10 cm² in size, removing most subcutaneous fat. These pieces were then placed in plastic zip-lock bags and kept on ice until the collection was complete. For long-term storage, all samples were stored at −20 °C until needed.

### Procine Skin Wounding and Infection

One day before wounding, frozen porcine skin samples were thawed in a 4 °C refrigerator overnight. Excess fat tissue was then trimmed away using a scalpel. The skin was disinfected with 70% (v/v) ethanol and sectioned into discs using an 8 mm biopsy punch. The stratum corneum layer was separated from the epidermis layer by peeling. Each skin disc was then wounded by 15 to 20 insertions with a 27G needle. Subsequently, 10 μL of 1×PBS containing 1×10⁶ CFU of S. aureus 25923 in the log phase were inoculated onto the wounded skin samples. Control groups, including those with wounds but no infection and those without any wounding, were also prepared. Each sample was placed onto 6% (w/v) agar to maintain skin moisture and incubated at 37 °C to establish infection. After 24 hours, 40 μL of NP formulations or 1×PBS was topically applied to the wounds. The samples were then returned to the incubator at 37 °C for an additional 24 hours.

### Procine Skin Histology

Skin samples were washed once with 1×PBS solution and fixed with a 2% (v/v) glutaraldehyde, 2% (v/v) paraformaldehyde, in 1×PBS solution at 4 °C for 24 hours. Subsequently, fixed samples were washed with 1×PBS once and dehydrated through a graded ethanol series (50%, 75%, 90%, and 100% (v/v)), before undergoing hematoxylin and eosin (H&E) staining and Gram staining. All sections were imaged via light microscope and analyzed using ImageJ (version 1.51).

### Procine Skin SEM Imaging

Skin samples were washed once with 1×PBS solution and fixed with a 2% (v/v) glutaraldehyde, 2% (v/v) paraformaldehyde, in 1×PBS solution at 4 °C for 24 hours. Samples were airdried overnight at room temperature, mounted on aluminium stubs with carbon tape, and sputter-coated gold-palladium was sputter-coated at 20 mA for 60 seconds with a sputter coater. Samples were then imaged with an SEM operated at 5 kV.

### In Vivo Antibiofilm Efficacy Studies

Subcutaneous infection murine model: Healthy male and female BALB/cJ mice, aged 6-8 weeks, were utilized in studies as they have been reported to have a similar immunological response to humans to S. aureus. See Singh, A., Amiji, M. M. Fundamentals of stimuli-responsive drug and gene delivery system. Biomaterials Science Series, No. 1, 2018. Full-thickness excisional wounds were achieved using a 6-mm-diameter biopsy punch, as previously reported. See Wang, X., Ge, J.; Tredget, E. E., Wu, Y. The mouse excisional wound splinting model, including applications for stem cell transplantation. Nature Protocols, 2013, 8, 302-309; Xu, W., Dielubanza, E., Maisel, A., Leung, K., Mustoe, T., Hong, S., Galiano, R. Staphylococcus aureus impairs cutaneous wound healing by activating the expression of a gap junction protein, connexin-43 in keratinocytes. Cellular and Molecular Life Sciences, 2021, 78, 935-947; Liu, M., Wang, Y., He, W., Liu, T., Zhou, D., Hu, X., Xing, M., Wu, J. Optimization and integration of nanosilver on polycaprolactone nanofibrous mesh for bacterial inhibition and wound healing in vitro and in vivo. International Journal of Nanomedicine, 2017, 12, 6827-6840; Anderson, L. S., Reynolds, M. B., Rivara, K. R., Miller, L. S., Simon, S. I. A Mouse Model to Assess Innate Immune Response to Staphylococcus aureus Infection. J. Vis. Exp, 2019 Feb. 28, 144. 10 mice per group (5 males, 5 females) were tested as this group size is reported to be sufficient for statistical significance in this model. See Xu, W., Dielubanza, E., Maisel, A., Leung, K., Mustoe, T., Hong, S., Galiano, R. Staphylococcus aureus impairs cutaneous wound healing by activating the expression of a gap junction protein, connexin-43 in keratinocytes. Cellular and Molecular Life Sciences, 2021, 78, 935-947; Coleman, D. C., Morici, L. A., Roy, C. J., Jp, H., Jk, F., Wang, Y., Jb, M., Mc, S., La, M., Cj, R., Hoffmann, J. P., Friedman, J. K., Wang, Y., Mclachlan, J. B., Sammarco, M. C. In situ Treatment with Novel Microbiocide Inhibits Methicillin Resistant Staphylococcus aureus in a Murine Wound Infection Model. Front Microbiol, 2020, 10:3106. In brief, mice were anaesthetized with isoflurane, and their dorsal skin was depilated and sanitized. Buprenorphine (0.05 mg/kg) was administered prophylactically for pain relief twice daily for two days post-wounding. One full-thickness wound was created in the depilated area using a 5-mm diameter sterile biopsy punch. See Wang, X., Ge, J., Tredget, E. E., Wu, Y. The mouse excisional wound splinting model, including applications for stem cell transplantation. Nature Protocols, 2013, 8, 302-309. 20 μL of S. aureus Xen29 (derived from parental strain S. aureus RN4220) with 1×10⁶ CFU was inoculated to each wounded site via pipetting directly into the wound beds. Following bacterial inoculation of the wounds, a transparent dressing (Tegaderm) was applied over wound areas. According to their experimental groups, mice are treated daily for seven days starting on day one post-infection. Treatments include a sterile 1×PBS control, GNPs, Ag-GNPs, DNase, and R-GNPs. Treatments were administered via topical application directly onto the wound site. Bacterial burdens were assessed daily via the in vivo imaging system (IVIS). Temperature and weight were recorded daily to monitor the overall health of the animals.

### Wound SEM Imaging

Wounds from the treatment study were fixed in 4% (v/v) formaldehyde for 2 hours at room temperature. Samples were washed twice in 1×PBS and fixed with 8% (v/v) formaldehyde in PBS at 4 °C for another 72 hours. Fixed wounds were then washed in 1×PBS. The tissues were then dehydrated with increasing concentrations of ethanol (20, 30, 40, 50, 60, 70, 90, and 100% (v/v) ethanol) with 10 min per gradient. Tissues were chemically dried in hexamethyldisilazane (HMDS) for 10 min and air-dried in a desiccator overnight. The samples were mounted on aluminium stubs with carbon tape and sputter-coated with 2 nm high-resolution platinum. Samples were air-dried overnight at room temperature and gold-palladium was sputter-coated at 20 mA for 60 s with a sputter coater (Jeol JFC-1600). Samples were then imaged with a SEM (Quattro S, Thermo Fisher Scientific, USA) operated at 2 kV.

### Wound Histology

Wound tissues were harvested at the end of the study and fixed with 4% (v/v) formaldehyde followed by overnight incubation in 70% (v/v) ethanol. Fixed tissue was processed and embedded into paraffin blocks, and μm thick sections were cut using a microtome (Leica RM2265). The sections were stained with either Hematoxylin and eosin stain (H&E) or Gram stain following manufacturing instructions. All sections were imaged via light microscope (Nikon Eclipse 50i) and analyzed using ImageJ (version 1.51). Immunofluorescence for macrophages using the primary antibody RM0029-11H3 (sc-101447, Santa Cruz, TX). Alexa 568 anti-rat IgG (Life Technologies, CA) was employed as the secondary antibody.

### Macroscopic Analysis of Wound Healing

Digital photographs of the wounds were taken daily (day 0-7) and wound closure was assessed via ImageJ. Briefly, each image was taken with a ruler as a reference and each measurement was calibrated with the reference. The wound surface area was calculated and % wound closure was calculated using Equation (4).

$$Wound closure ( \% ) = \frac{measured wound size}{initial wound size} \times 100 \qquad ( 4 )$$

### Statistical Analysis

All experiments were conducted in triplicate at a minimum. Statistical significance was calculated using GraphPad Prism™ with Analysis of variance (ANOVA) with Tukey's posthoc analysis or two-way ANOVA with multiple comparisons with Dunnett's test. Statistical significance was represented as follows, *p<0.1, **p<0.01, ***p<0.001, and ****p<0.0001. Additional Material and Methods

### Monitoring of the Biofilm Stimuli

The eDNA component of the biofilm matrix was visualized using 1 μM of 7-hydroxy-9H-(1,3-dichloro-9,9-dimethylacridin-2-one) (DDAO), a red fluorescent probe (AAT Bioquest, CA, USA; excitation at 663 nm and emission at 660 nm). See Tahrioui, A. et al. Extracellular DNA release, quorum sensing, and PrrF1/F2 small RNAs are key players in Pseudomonas aeruginosa tobramycin-enhanced biofilm formation. NPJ Biofilms Microbiomes, 2019, 5 (1): 15; Ren, P., Chen, T., Liu, N., Sun, W., Hu, G., Yu, Y., Yu, B., Ouyang, P., Liu, D., Chen, Y. Efficient Biofilm-Based Fermentation Strategies by eDNA Formation for 1-Proline Production with Corynebacterium glutamicum. ACS Omega, 2020, 5, 33314-33322. A confocal laser scanning microscope (CLSM, A1R confocal laser microscope, Nikon Instruments, Inc., Melville, NY) with an Apo LWD 25×1.10 W water immersion objective was utilized to visualize the biofilms and the eDNA content. Briefly, biofilms were formed in 96 well glass bottom plate as previously described. Biofilm samples were washed with 1×PBS twice. Hoechst Stains 33342 (0.6 μg/mL) (Thermo Fisher Scientific, USA) (See, Jurcisek, J. A.; Bakaletz, L. O. Biofilms formed by nontypeable Haemophilus influenzae in vivo contain both double-stranded DNA and type IV pilin protein. Journal of Bacteriology 2007, 189, 3868-3875) and DDAO fluorescent dyes (0.2 μg/mL) were added, allowed to settle, and used to stain the nuclei and eDNA for 20 min. The slides were then washed three times with 1×PBS and the sample was immediately observed under the CLSM.

Biofilm pHs were measured using a pH probe (Hanna Instruments, USA). Biofilm samples were prepared as mentioned previously. The biofilm was disrupted by pipetting, and a pH probe was inserted into the solution and allowed to equilibrate for 5 minutes before recording the pH value. The pH meter was calibrated before each measurement using pH 4, 7 and 10 buffer solutions. Three independent measurements were taken for each sample, and the mean value with standard deviation was reported. The entire experiment was conducted at room temperature (22-25 °C) to avoid any temperature-related effects on pH.

Bacterial gelatinase production was confirmed using the gelatin hydrolysis assay.4 Briefly, LB-gelatin (3%, w/v) gel was prepared in culture tubes (4 mL per tube) and kept on ice until the gel was solidified. Bacteria overnight culture is stab-inoculated into the LB-gelatin gel and incubated at 37 °C for up to 7 days. The gels were monitored daily for gelatin liquefaction. Gelatinase enzymatic activity can be visually assessed after ice bath incubation since hydrolyzed gelatin is unable to solidify (gelatin liquefies at >28 °C, solidifies at <4 °C). Thus, the nutrient gelatin tubes are incubated in an ice bath for 30 min to confirm if liquefication was due to enzyme activity.

### Confirmation of Coated DNase Activity Via Gel Electrophoresis

The hydrolysis ability of NPs against biofilms was analysed by treating S. aureus 24-hour aged biofilm with different concentrations of P-D-GNPs. After treatment, biofilms were disrupted via vortexing and sonication, cells were separated from the EPS by centrifugation (5000×g for 10 min at 4 °C. The supernatants were collected and filtered through 0.2 μm cellulose acetate filter. The extracted eDNA was visualized with gel electrophoresis as described above.

### Characterization of Biofilm Environmental pH

Bacterial cultures were prepared according to previously described methods. Biofilms were cultivated in 24-well tissue culture-treated plates coated with human blood plasma. The biofilms were disrupted by pipetting, and supernatants were collected via centrifugation at 5000 xg for 10 minutes at 4 °C. Before each measurement, the pH meter (Mettler Toledo) was calibrated with buffer solutions at pH 4, 7, and 10 (Thermo Fisher Scientific). The pH probe was immersed in each sample and allowed to stabilize for 2-3 minutes before the pH value was recorded. For each condition, three independent measurements were performed, and the results were expressed as the mean±standard deviation. All experiments were carried out at room temperature (22-25 °C) to mitigate any temperature-related effects on the pH readings.

### Minimum Inhibitory and Bactericidal Concentration Assay

Each strain was seeded at a final concentration of 1×10⁶ CFU/mL in their exponential growth phase (Optical density at 600 nm (OD₆₀₀)=0.1 (OD₆₀₀=0.1)) and added to a plate containing serially diluted NP therapeutics. Positive controls (PC) (only bacteria, no treatment), negative controls (NC) (media only), therapeutic controls (TC) (AgNPs and DNase alone), and therapeutic negative controls (TNC) (media with therapeutics) were included. Samples were incubated at 37 °C for 16-18 hours with orbital shaking at 200 rpm. OD₆₀₀ is measured, and normalized bacteria growth is calculated using Equation (5).

$$Normalized growth ( \% ) = \frac{{OD}_{600 , sample} - {OD}_{600 , T NC}}{{OD}_{600 , PC} - {OD}_{600 , NC}} \times 100 \qquad ( 5 )$$

The minimal bactericidal concentration (MBC) was assessed by plating 10 μL from each treatment group on 1.5% (w/v) nutrient agar plates and incubating at 37 °C for 16-18 hours. The lowest therapeutic concentration at which no visible colony formation is taken as the MBC of the NPs against tested bacterial strains.

### Biofilm Inhibition Assay

The effect of the NPs on biofilm formation was analyzed via CV staining. Briefly, biofilms were formed as previously described with the concurrent addition of treatments. Treatments will be introduced at a concentration range of 0.1×MIC to 100×MIC value. As previously mentioned, PC (positive control), NC (negative control), TC (therapeutic control), and TNC (therapeutic negative control) were included. After 24 h incubation at 37 °C, the supernatant was removed, and biofilms were washed with 1×PBS. The biomass was washed once with 1×PBS, fixed with 100% methanol and stained with a 0.1% (w/v) CV solution. The CV solution was removed post staining, and each well was washed 3 times with 1×PBS and eluted with 100% methanol. The OD of the CV stain was measured at 590 nm (OD₅₉₀), where a higher absorbance reading corresponds with greater biomass. The percentage of remaining biofilm biomass was quantified using Equation (6). The lowest drug concentration that inhibits bacteria biofilm growth is taken as the minimal biofilm inhibitory concentration (MBIC).

$$Normalized biofilm biomass ( \% ) = \frac{{OD}_{550 , sample} - {OD}_{550 , drug , NC} - {OD}_{550 , NC}}{{OD}_{550 , PC} - {OD}_{550 , NC}} \qquad ( 6 )$$

### Example XII

### Embodiments

Embodiments of the present disclosure are directed to a method of delivering an antimicrobial agent to microbes within a biofilm including contacting the biofilm with a nanoparticle including (1) a core comprising gelatin and an antimicrobial agent, (2) an antibiofilm agent attached to the core, and a (3) pH-degradable material attached to the core, wherein the core is responsive to a gelatinase where the gelatinase degrades the core to release the antimicrobial agent wherein the antimicrobial agent is delivered to the microbes, and wherein the pH-degradable material is responsive to a low pH to degrade the pH-degradable material, and wherein the antibiofilm agent degrades the biofilm. According to one aspect, the antibiofilm agent comprises a nuclease, a polysaccharide hydrolase, and/or a protease. According to one aspect, the nuclease is deoxyribonuclease I (DNase I). According to one aspect, the pH-degradable material is a poly(ethylene glycol) (PEG) derivative. According to one aspect, the pH-degradable material is methoxy PEG-benzoic-imine-branched polyethyleneimine (mPEG-b-i-bPEI). According to one aspect, the antimicrobial agent comprises an antibiotic, an antibacterial agent, or an antifungal agent, or a combination thereof. According to one aspect, the microbes include a bacteria or a fungus, or a combination of a bacteria and a fungus.

Embodiments of the present disclosure are directed to a nanoparticle including a core including gelatin and an antimicrobial agent, an antibiofilm agent attached to the core, and a pH-degradable material attached to the core. According to one aspect, the antibiofilm agent includes a nuclease, a polysaccharide hydrolase, and/or a protease. According to one aspect, the nuclease is deoxyribonuclease I (DNase I). According to one aspect, the antimicrobial agent includes an antibiotic, an antibacterial agent, or an antifungal agent, or a combination thereof. According to one aspect, the pH-degradable material is a poly(ethylene glycol) (PEG) derivative. According to one aspect, the pH-degradable material is methoxy PEG-benzoic-imine-branched polyethyleneimine (mPEG-b-i-bPEI).

Embodiments of the present disclosure are directed to a pharmaceutical composition including a nanoparticle including a core including gelatin and an antimicrobial agent, an antibiofilm agent attached to the core, and a pH-degradable material attached to the core, and a pharmaceutically acceptable excipient.

Embodiments of the present disclosure are directed to a method of treating a subject in need thereof for a microbial infection including administering to the subject a nanoparticle including a core including gelatin and an antimicrobial agent, an antibiofilm agent attached to the core, and a pH-degradable material attached to the core, wherein the microbial infection is prevented, reduced, inhibited or eradicated. According to one aspect, the nanoparticle is administered by systemic administration or topically. According to one aspect, the subject is a mammal. According to one aspect, the subject is a human. According to one aspect, the microbial infection is a bacterial infection, a fungal infection, or a combination of bacterial and fungal infection. According to one aspect, the microbial infection is characterized by a bacteria or a fungus or a combination of a bacteria and a fungus.

Embodiments of the present disclosure are directed to a method of making a nanoparticle including providing a gelatin core including an antimicrobial agent, attaching an antibiofilm agent to the gelatin core, and attaching a pH-degradable material to the gelatin core. According to one aspect, the antibiofilm agent includes a nuclease, a polysaccharide hydrolase, and/or a protease. According to one aspect, the nuclease is deoxyribonuclease I (DNase I). According to one aspect, the antimicrobial agent includes an antibiotic, an antibacterial agent, or an antifungal agent, or a combination thereof. According to one aspect, the pH-degradable material is a poly(ethylene glycol) (PEG) derivative. According to one aspect, the pH-degradable material is methoxy PEG-benzoic-imine-branched polyethyleneimine (mPEG-b-i-bPEI).

Embodiments of the present disclosure are directed to a method of delivering an antibiotic to bacteria within a biofilm including contacting the biofilm with a nanoparticle including (1) a core comprising gelatin and an antibiotic, (2) a nuclease attached to the core, and a (3) pH-degradable material attached to the core, wherein the core is responsive to a gelatinase where the gelatinase degrades the core to release the antibiotic wherein the antibiotic is delivered to the bacteria, and wherein the pH-degradable material is responsive to a low pH to degrade the pH-degradable material, and wherein the nuclease degrades extracellular DNA of the biofilm. According to one aspect, the nuclease is DNase I. According to one aspect, the pH-degradable material is a poly(ethylene glycol) (PEG) derivative. According to one aspect, the pH-degradable material is methoxy PEG-benzoic-imine-branched polyethyleneimine (mPEG-b-i-bPEI).

Embodiments of the present disclosure are directed to a nanoparticle including a core comprising gelatin and an antibiotic, a nuclease attached to the core, and a pH-degradable material attached to the core. According to one aspect, the nuclease is DNase I. According to one aspect, the pH-degradable material is a poly(ethylene glycol) (PEG) derivative. According to one aspect, the pH-degradable material is methoxy PEG-benzoic-imine-branched polyethyleneimine (mPEG-b-i-bPEI).

Embodiments of the present disclosure are directed to a pharmaceutical composition comprising a nanoparticle including a core including gelatin and an antibiotic, a nuclease attached to the core, and a pH-degradable material attached to the core, and a pharmaceutically acceptable excipient. According to one aspect, the nuclease is DNase I. According to one aspect, the pH-degradable material is a poly(ethylene glycol) (PEG) derivative. According to one aspect, the pH-degradable material is methoxy PEG-benzoic-imine-branched polyethyleneimine (mPEG-b-i-bPEI).

Embodiments of the present disclosure are directed to a method of treating a subject in need thereof for a bacterial infection including administering to the subject a nanoparticle including a core including gelatin and an antibiotic, a nuclease attached to the core, and a pH-degradable material attached to the core, wherein the bacterial infection is prevented, reduced, inhibited or eradicated. According to one aspect, the nuclease is DNase I. According to one aspect, the pH-degradable material is a poly(ethylene glycol) (PEG) derivative. According to one aspect, the pH-degradable material is methoxy PEG-benzoic-imine-branched polyethyleneimine (mPEG-b-i-bPEI). According to one aspect, the nanoparticle is administered by systemic administration or topically. According to one aspect, the subject is a mammal. According to one aspect, the subject is a human.

Embodiments of the present disclosure are directed to a method of making a nanoparticle including providing a gelatin core including an antibiotic, attaching a nuclease to the gelatin core, and attaching a pH-degradable material to the gelatin core. According to one aspect, the nuclease is DNase I. According to one aspect, the pH-degradable material is a poly(ethylene glycol) (PEG) derivative. According to one aspect, the pH-degradable material is methoxy PEG-benzoic-imine-branched polyethyleneimine (mPEG-b-i-bPEI).

Embodiments of the present disclosure are directed to a method of delivering an antibiotic to bacteria within a biofilm including contacting the biofilm with a nanoparticle including (1) a core comprising gelatin and an antibiotic, (2) an antibiofilm agent attached to the core, and a (3) pH-degradable material attached to the core, wherein the core is responsive to a gelatinase where the gelatinase degrades the core to release the antibiotic wherein the antibiotic is delivered to the bacteria, and wherein the pH-degradable material is responsive to a low pH to degrade the pH-degradable material, and wherein the antibiofilm agent degrades the biofilm. In some embodiments, the antibiofilm agent includes a nuclease, a polysaccharide hydrolase, and/or a protease. In one embodiment, the nuclease is deoxyribonuclease I (DNase I). In one embodiment, the pH-degradable material is a poly(ethylene glycol) (PEG) derivative. In another embodiment, the pH-degradable material is methoxy PEG-benzoic-imine-branched polyethyleneimine (mPEG-b-i-bPEI).

Embodiments of the present disclosure are directed to a nanoparticle including a core comprising gelatin and an antibiotic, an antibiofilm agent attached to the core, and a pH-degradable material attached to the core. In one embodiment, the antibiofilm agent includes a nuclease, a polysaccharide hydrolase, and/or a protease. In one embodiment, the nuclease is deoxyribonuclease I (DNase I). In one embodiment, the pH-degradable material is a poly(ethylene glycol) (PEG) derivative. In another embodiment, the pH-degradable material is methoxy PEG-benzoic-imine-branched polyethyleneimine (mPEG-b-i-bPEI).

Embodiments of the present disclosure are directed to a pharmaceutical composition including (1) a core comprising gelatin and an antibiotic, (2) an antibiofilm agent attached to the core, and a (3) pH-degradable material attached to the core, and a pharmaceutically acceptable excipient.

Embodiments of the present disclosure are directed to a method of treating a subject in need thereof for a bacterial infection including administering to the subject a nanoparticle including (1) a core comprising gelatin and an antibiotic, (2) an antibiofilm agent attached to the core, and a (3) pH-degradable material attached to the core, wherein the bacterial infection is prevented, reduced, inhibited or eradicated. In one embodiment, the nanoparticle is administered by systemic administration or topically. In one embodiment, the subject is a mammal. In one embodiment, the subject is a human.

Embodiments of the present disclosure are directed to a method of making a nanoparticle including providing a gelatin core including an antibiotic, attaching an antibiofilm agent to the gelatin core, and attaching a pH-degradable material to the gelatin core.

Embodiments of the present disclosure are directed to a method of delivering an antimicrobial agent to microbes within a biofilm including contacting the biofilm with a nanoparticle including (1) a core comprising gelatin and an antimicrobial agent, (2) an antibiofilm agent attached to the core, and a (3) pH-degradable material attached to the core, wherein the core is responsive to a gelatinase where the gelatinase degrades the core to release the antimicrobial agent wherein the antimicrobial agent is delivered to the microbes, and wherein the pH-degradable material is responsive to a low pH to degrade the pH-degradable material, and wherein the antibiofilm agent degrades the biofilm. In one embodiment, the antibiofilm agent comprises a nuclease, a polysaccharide hydrolase, and/or a protease. In another embodiment, the nuclease is deoxyribonuclease I (DNase I). In one embodiment, the pH-degradable material is a poly(ethylene glycol) (PEG) derivative. In another embodiments, the pH-degradable material is methoxy PEG-benzoic-imine-branched polyethyleneimine (mPEG-b-i-bPEI). In one embodiment, the antimicrobial agent comprises an antibiotic, an antibacterial agent, and/or an antifungal agent.

Embodiments of the present disclosure are directed to a nanoparticle including a core including gelatin and an antimicrobial agent, an antibiofilm agent attached to the core, and a pH-degradable material attached to the core. In one embodiment, the antibiofilm agent includings a nuclease, a polysaccharide hydrolase, and/or a protease. In another embodiment, the nuclease is deoxyribonuclease I (DNase I). In one embodiment, the antimicrobial agent comprises an antibiotic, an antibacterial agent, and/or an antifungal agent. In one embodiment, the pH-degradable material is a poly(ethylene glycol) (PEG) derivative. In another embodiment, the pH-degradable material is methoxy PEG-benzoic-imine-branched polyethyleneimine (mPEG-b-i-bPEI).

Embodiments of the present disclosure are directed a pharmaceutical composition including a nanoparticle including (1) a core comprising gelatin and an antimicrobial agent, (2) an antibiofilm agent attached to the core, and a (3) pH-degradable material attached to the core, and a pharmaceutically acceptable excipient.

Embodiments of the present disclosure are directed a method of treating a subject in need thereof for a microbial infection including administering to the subject a nanoparticle of claim 37, wherein the microbial infection is prevented, reduced, inhibited or eradicated. In one embodiment, the nanoparticle is administered by systemic administration or topically. In one embodiment, the subject is a mammal. In one embodiment, the subject is a human. In one embodiment, the microbial infection is a bacterial infection, a fungal infection, or a combination of bacterial and fungal infection.

Embodiments of the present disclosure are directed a method of making a nanoparticle including providing a gelatin core including an antimicrobial agent, attaching an antibiofilm agent to the gelatin core, and attaching a pH-degradable material to the gelatin core.

1. A method of delivering an antimicrobial agent to microbes within a biofilm comprising
contacting the biofilm with a nanoparticle comprising
(1) a core comprising gelatin and an antimicrobial agent,
(2) an antibiofilm agent attached to the core, and a
(3) pH-degradable material attached to the core,
wherein the core is responsive to a gelatinase where the gelatinase degrades the core to release the antimicrobial agent wherein the antimicrobial agent is delivered to the microbes, and
wherein the pH-degradable material is responsive to a low pH to degrade the pH-degradable material, and
herein the antibiofilm agent degrades the biofilm.

2. The method of claim 1 wherein the antibiofilm agent comprises a nuclease, a polysaccharide hydrolase, and/or a protease.

3. The method of claim 2 wherein the nuclease is deoxyribonuclease I (DNase I).

4. The method of claim 1 wherein the pH-degradable material is a poly(ethylene glycol) (PEG) derivative.

5. The method of claim 1 wherein the pH-degradable material is methoxy PEG-benzoic-imine-branched polyethyleneimine (mPEG-b-i-bPEI).

6. The method of claim 1 wherein the antimicrobial agent comprises an antibiotic, an antibacterial agent, or an antifungal agent, or a combination thereof.

7. A nanoparticle comprising
a core comprising gelatin and an antimicrobial agent,
an antibiofilm agent attached to the core, and a
pH-degradable material attached to the core.

8. The nanoparticle of claim 7 wherein the antibiofilm agent comprises a nuclease, a polysaccharide hydrolase, and/or a protease.

9. The nanoparticle of claim 8 wherein the nuclease is deoxyribonuclease I (DNase I).

10. The nanoparticle of claim 7 wherein the antimicrobial agent comprises an antibiotic, an antibacterial agent, or an antifungal agent or a combination thereof.

11. The nanoparticle of claim 7 wherein the pH-degradable material is a poly(ethylene glycol) (PEG) derivative.

12. The nanoparticle of claim 7 wherein the pH-degradable material is methoxy PEG-benzoic-imine-branched polyethyleneimine (mPEG-b-i-bPEI).

13. A pharmaceutical composition comprising a nanoparticle of claim 7 and a pharmaceutically acceptable excipient.

14. A method of treating a subject in need thereof for a microbial infection comprising
administering to the subject a nanoparticle of claim 7, wherein the microbial infection is prevented, reduced, inhibited or eradicated.

15. The method of claim 14 wherein the nanoparticle is administered by systemic administration or topically.

16. The method of claim 14 wherein the subject is a mammal.

17. The method of claim 14 wherein the subject is a human.

18. The method of claim 14 wherein the microbial infection is a bacterial infection, a fungal infection, or a combination of a bacterial and a fungal infection.

19. A method of making a nanoparticle comprising
providing a gelatin core including an antimicrobial agent,
attaching an antibiofilm agent to the gelatin core, and
attaching a pH-degradable material to the gelatin core.

20. The method of claim 1 wherein the microbes include a bacteria or a fungus or a combination of a bacteria and a fungus.

21. The method of claim 14 wherein the microbial infection is characterized by a bacteria or a fungus or a combination of a bacteria and a fungus.

22. The method of claim 19 wherein the antibiofilm agent comprises a nuclease, a polysaccharide hydrolase, and/or a protease.

23. The method of claim 22 wherein the nuclease is deoxyribonuclease I (DNase I).

24. The method of claim 19 wherein the antimicrobial agent comprises an antibiotic, an antibacterial agent, or an antifungal agent, or a combination thereof.

25. The method of claim 19 wherein the pH-degradable material is a poly(ethylene glycol) (PEG) derivative.

26. The method of claim 19 wherein the pH-degradable material is methoxy PEG-benzoic-imine-branched polyethyleneimine (mPEG-b-i-bPEI).
